(19) United States
(12) Patent Application Publication
(10) Pub. No.: US 2020/0020444 A1
(43) Pub. Date: Jan. 16, 2020

(54) INFORMATION PROCESSING DEVICE, INFORMATION PROCESSING METHOD, AND PROGRAM

(71) Applicant: SONY CORPORATION, TOKYO (JP)

(72) Inventor: SHO FURUICHI, TOKYO (JP)

(21) Appl. No.: 16/334,889

(22) PCT Filed: Sep. 15, 2017
(86) PCT No.: PCT/JP2017/033424

§ 371 Date: Mar. 20, 2019

(30) Foreign Application Priority Data
Oct. 20, 2016 (JP) 2016-206245

(51) Int. Cl. G16H 40/67; H04L 29/06

(52) U.S. Cl. CPC G16H 40/67; H04L 63/08

(57) ABSTRACT

[Object] To manage authority to execute a process in a more suitable manner even in a situation in which a process associated with medical care is executed via a network. [Solution] An information processing device, including: a receiving unit configured to receive a request related to execution of a process associated with medical care from a device directly or indirectly connected via a network; and a control unit configured to control a setting of authority related to execution of the process for a device which is caused to execute the process corresponding to the request or a user associated with the device in accordance with attribute information directly or indirectly associated with the device.

### TECHNICAL FIELD

The present disclosure relates to an information processing device, an information processing method, and a program.

### BACKGROUND ART

In recent years, with the development of communication technology, in the medical field, for example, in order to realize provision of medical services to a remote place such as in telemedicine and cooperation of a plurality of medical institutions, the use of a network such as the Internet has been variously reviewed. For example, an example of a technique for linking different medical information systems via a network is disclosed in Patent Literature 1.

### CITATION LIST

### Patent Literature

Patent Literature 1: JP 2015-28773A

### DISCLOSURE OF INVENTION

### Technical Problem

Meanwhile, an appropriate license or qualification is necessary for actions related to medical care (hereinafter, also referred to collectively as “medical practice”), for example, a doctor's license is necessary for conducting actions such as medical examination or diagnosis, and a nursing license is necessary for actions for assisting with a medical examination or the like. Further, in the field of medical care, information with high confidentiality such as patient information is often handled, and it is important to secure confidentiality of various types of information. This is similar, for example, even in a case in which a medical service is provided via a network or in a case in which information is transmitted between a plurality of medical institutions. Under such circumstances, in a case in which communication technology is applied to the medical field, management of authority to execute a process associated with the medical care such as implementation of medical practice and management of various types of information (for example, browsing of information) has become more important.

In this regard, the present disclosure proposes an information processing device, an information processing method, and a program which are capable of managing authority to execute a process in a more suitable manner even in a situation in which a process associated with medical care is executed via a network.

### Solution to Problem

According to the present disclosure, there is provided an information processing device, including: a receiving unit configured to receive a request related to execution of a process associated with medical care from a device directly or indirectly connected via a network; and a control unit configured to control a setting of authority related to execution of the process for a device which is caused to execute the process corresponding to the request or a user associated with the device in accordance with attribute information directly or indirectly associated with the device.

In addition, according to the present disclosure, there is provided an information processing device, including: a transmitting unit configured to transmit a request related to execution of a process associated with medical care to a device directly or indirectly connected via a network; and an acquiring unit configured to acquire a control result of a setting of authority associated with execution of the process for the device or a user associated with the device, corresponding to attribute information directly or indirectly associated with the device which is caused to execute the process corresponding to the request after the request is transmitted.

In addition, according to the present disclosure, there is provided an information processing device, including: a receiving unit configured to receive a request related to authentication of a process associated with medical care from a device directly or indirectly connected via a network; and an authentication processing unit configured to execute the authentication on an authentication target corresponding to the request in accordance with attribute information directly or indirectly associated with the authentication target.

In addition, according to the present disclosure, there is provided an information processing device, including: a transmitting unit configured to transmit a request related to authentication of a process associated with medical care to a device directly or indirectly connected via a network; and an acquiring unit configured to acquire a result of the authentication on an authentication target corresponding to the request in accordance with attribute information directly or indirectly associated with the authentication target from the device after the request is transmitted.

In addition, according to the present disclosure, there is provided an information processing method, including: receiving, by a computer, a request related to execution of a process associated with medical care from a device directly or indirectly connected via a network; and controlling, by the computer, a setting of authority related to execution of the process for a device which is caused to execute the process corresponding to the request or a user associated with the device in accordance with attribute information directly or indirectly associated with the device.

In addition, according to the present disclosure, there is provided an information processing method, including: transmitting, by a computer, a request related to execution of a process associated with medical care to a device directly or indirectly connected via a network; and acquiring, by the computer, a control result of a setting of authority associated with execution of the process for the device or a user associated with the device, corresponding to attribute information directly or indirectly associated with the device which is caused to execute the process corresponding to the request from the device after the request is transmitted.

In addition, according to the present disclosure, there is provided an information processing method, including: receiving, by a computer, a request related to authentication of a process associated with medical care from a device directly or indirectly connected via a network; and executing, by the computer, the authentication on an authentication target corresponding to the request in accordance with attribute information directly or indirectly associated with the authentication target.

In addition, according to the present disclosure, there is provided an information processing method, including: transmitting, by a computer, a request related to authentication of a process associated with medical care to a device directly or indirectly connected via a network; and acquiring, by the computer, a result of the authentication on an authentication target corresponding to the request in accordance with attribute information directly or indirectly associated with the authentication target from the device after the request is transmitted.

In addition, according to the present disclosure, there is provided a program causing a computer to execute: receiving a request related to execution of a process associated with medical care from a device directly or indirectly connected via a network; and controlling a setting of authority related to execution of the process for a device which is caused to execute the process corresponding to the request or a user associated with the device in accordance with attribute information directly or indirectly associated with the device.

In addition, according to the present disclosure, there is provided a program causing a computer to execute: transmitting a request related to execution of a process associated with medical care to a device directly or indirectly connected via a network; and acquiring a control result of a setting of authority associated with execution of the process for the device or a user associated with the device, corresponding to attribute information directly or indirectly associated with the device which is caused to execute the process corresponding to the request from the device after the request is transmitted.

In addition, according to the present disclosure, there is provided a program causing a computer to execute: receiving a request related to authentication of a process associated with medical care from a device directly or indirectly connected via a network; and executing the authentication on an authentication target corresponding to the request in accordance with attribute information directly or indirectly associated with the authentication target.

In addition, according to the present disclosure, there is provided a program causing a computer to execute: transmitting a request related to authentication of a process associated with medical care to a device directly or indirectly connected via a network; and acquiring a result of the authentication on an authentication target corresponding to the request in accordance with attribute information directly or indirectly associated with the authentication target from the device after the request is transmitted.

### Advantageous Effects of Invention

As described above, according to the present disclosure, an information processing device, an information processing method, and a program which are capable of managing an authority to execute a process in a more suitable manner even in a situation in which a process associated with medical care is executed via a network are provided.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an explanatory diagram for describing an example of an architecture of an information processing system according to an embodiment of the present disclosure.

FIG. 2 is a block diagram illustrating an example of a functional configuration of a medical network manager according to the embodiment.

FIG. 3 is a block diagram illustrating an example of a functional configuration of a medical network endpoint according to the embodiment.

FIG. 4 is a sequence diagram illustrating an example of a flow of a series of processes for a procedure related to registration of information in an information processing system according to the embodiment.

FIG. 5 is a sequence diagram illustrating an example of a flow of a series of processes for a procedure related to registration of information in an information processing system according to the embodiment.

FIG. 6 is a sequence diagram illustrating an example of a flow of a series of processes for a procedure related to registration of information in an information processing system according to the embodiment.

FIG. 7 is a sequence diagram illustrating a flow of a series of processes for an example of a procedure related to control of setting of authority in an information processing system according to the embodiment.

FIG. 8 is a sequence diagram illustrating a flow of a series of processes for another example of a procedure related to control of setting of authority in an information processing system according to the embodiment.

FIG. 9 is a sequence diagram illustrating a flow of a series of processes for another example of a procedure related to control of setting of authority in an information processing system according to the embodiment.

FIG. 10 is an explanatory diagram for describing an overview of an information processing system according to a first example.

FIG. 11 is a sequence diagram illustrating an example of a flow of a series of processes of the information processing system according to the first example.

FIG. 12 is a sequence diagram illustrating an example of a flow of a series of processes of the information processing system according to the second example.

FIG. 13 is a sequence diagram illustrating an example of a flow of a series of processes of the information processing system according to the third example.

FIG. 14 is an explanatory diagram for describing an example a schematic configuration of an information processing system according to a fourth example.

FIG. 15 is a sequence diagram illustrating an example of a flow of a series of processes of the information processing system according to the fourth example.

FIG. 16 is an explanatory diagram for describing an example a schematic configuration of an information processing system according to a fifth example.

FIG. 17 is a sequence diagram illustrating an example of a flow of a series of processes of the information processing system according to the fifth example.

FIG. 18 is an explanatory diagram for describing an example a schematic configuration of an information processing system according to a sixth example.

FIG. 19 is a sequence diagram illustrating an example of a flow of a series of processes of the information processing system according to the sixth example.

FIG. 20 is a sequence diagram illustrating an example of a flow of a series of processes of the information processing system according to the sixth example.

FIG. 21 is a functional block diagram illustrating an example of a hardware configuration of an information processing device constituting an information processing system according to an embodiment.

### MODE(S) FOR CARRYING OUT THE INVENTION

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Further, the description will proceed in the following order.

### 1. Introduction

### 2. Architecture

3. Configuration examples
3.1. Configuration example of medical network manager
3.2. Configuration example of medium network endpoint
4. Technical features
4.1. Procedure related to registration of information
4.2. Procedure related to control of setting of authority

### 5. Examples

5.1. First example: doctor-patient matching service in remote diagnosis
5.2. Second example: remote control of medical device
5.3. Third example: handling authority authentication of powerful drugs
5.4. Fourth example: patient watching and handover by nurse
5.5. Fifth example: transfer of hospitalized patient
5.6. Sixth example: supervision and business permission for medical institution by supervising institution
6. Hardware configuration

### 7. Conclusion

### 1. INTRODUCTION

First, an overview of an information processing system according to an embodiment of the present disclosure will be described. A medical system according to the present embodiment provides a mechanism for realizing provision of medical services via a network such as telemedicine, cooperation of a plurality of medical institutions which are apart from each other via a network, and the like.

Further, in the present disclosure, for example, any of the following networks can be included as the network for providing medical services:
a medical institution intra network;
a network between medical institutions;
a network between a medical institution and doctors;
a network between doctors;
a network between a medical institution and nurses;
a network between nurses;
a network between a medical institution and patients;
a network between doctors and patients;
a network between nurses and patients;
a network between a medical institution and pharmacists (pharmacies);
a network between doctors and pharmacists (pharmacies);
a network between pharmacists (pharmacies); and
a network between pharmacists (pharmacies) and patients.

Further, the network for providing the medical services may include a plurality of networks among the various networks listed above. Further, a type of network for providing the medical services is not particularly limited as long as it can realize provision of medical services. Specifically, the network may be configured as a wired network, a wireless network, or a combination thereof.

Further, in a case in which at least a part of the network for providing the medical services is constituted as a wireless network, a frequency band to be used and a type of wireless technology are inconsequential. As a specific example, it may be a licensed band, a license-exempt band, or a light licensing band. Further, a light licensing band is a frequency band in which identifying and use permission of available frequencies are executed by a frequency management database as in a TV white space or a Citizens Broadband Radio Service specified in C.F.R. Part 96 in the United States. In other words, a frequency band which is primarily used by another service may be secondarily used for providing the medical services. As a specific example, unused channels included in a frequency band of digital TV broadcasting may be used as the network for providing the medical services. Further, for wireless technology, mobile communication technology (including GSM (registered trademark), UMTS, LTE, LTE-advanced, 5G, or later technology), wireless LAN technology (IEEE 802.11), a personal area network (PAN) (IEEE 802.15 or the like), a wireless regional area network (WRAN) (IEEE 802.22), or the like may be used.

Further, examples of the medical service include telemedicine, or watching over patients (for example, hospitalized patients) in hospitals, nursing homes, or the like. Here, telemedicine involves actions related to medical care such as diagnosis and medical treatment and actions related to health such as home health management that are conducted using communication technology without directly meeting with the patient. Examples of telemedicine include telepathology, teleradiology, and telecare. In telepathology, a doctor at a remote site performs pathology diagnosis via a network. Further, in teleradiology, a doctor at a remote site performs diagnosis for images which are captured with X-rays, computed tomography (CT), magnetic resonance imaging (MRI), or the like and transferred from another medical institution via a network. Further, in telecare, a doctor at a remote site provides guidance related to health management to a patient on the basis of a measurement result (for example, blood pressure, an electrocardiogram, or the like) of a health state of the patient by a health management terminal which is transmitted via a network. Further, in the present disclosure, in a case in which “medical service” is described, for the sake of convenience, it is not limited to so-called medical services such as telemedicine or watching over a patient but is assumed to also include various types of procedures or processes related to medical practice.

Incidentally, in a case in which provision of medical services or cooperation between medical institutions is realized using so-called information technology (IT) such as communication technology, “securing of confidentiality of information,” “management of authority related to medical care, nursing, or the like,” “prompt transmission of information,” and the like are sometimes required.

In particular, in a case in which IT such as communication technology is applied to the field of medical care, management of authority to execute a process associated with medical care such as implementation of medical practice and management of various types of information (browsing or the like) has become more important. For example, an appropriate license or qualification is necessary for medical care, for example, a doctor's license is necessary for conducting actions such as medical examination or diagnosis, and a nursing license is necessary for actions for assisting with a medical examination or the like. Further, in the field of medical care, information with high confidentiality such as patient information is often handled, and it is important to secure confidentiality of various types of information. This is similar, for example, even in a case in which a medical service is provided via a network or in a case in which information is transmitted between a plurality of medical institutions.

In light of such a situation, the present disclosure proposes an example of technology capable of realizing management of setting of authority such as granting, delegation, or the like of authority to execute a process associated with medical care even in a situation in which the process is executed via a network.

### 2. ARCHITECTURE

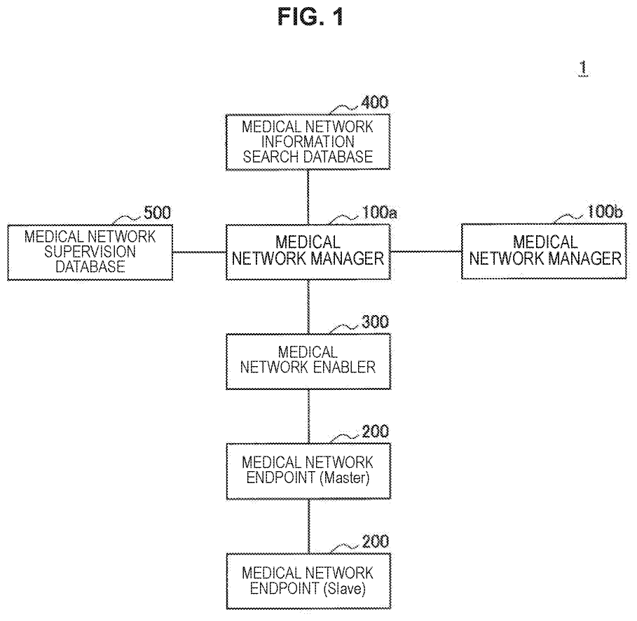

First, an example of an architecture of an information processing system according to the present embodiment will be described with reference to FIG. 1. FIG. 1 is an explanatory diagram for describing an example of an architecture of an information processing system according to the present embodiment.

As illustrated in FIG. 1, an information processing system 1 according to the present embodiment includes a medical network manager 100, a medical network endpoint 200, a medical network enabler 300, and a medical network information search database 400. The information processing system 1 may further include a medical network supervision database 500.

The medical network manager 100 is an entity responsible for various types of decision making related to provision of medical services via a network. As a specific example, the medical network manager 100 manages settings of authority of various types of processes (for example, medical practice (for example, medical examination, diagnosis, nursing, prescription, and the like) and a process related to an information browsing action or the like) on the medical network endpoint 200 to be described later or a user (for example, a doctor, a nurse, or the like) associated with the medical network endpoint 200. As a more specific example, the medical network manager 100 may grant authority to execute the process associated with the medical practice to the medical network endpoint 200 associated with the doctor or the nurse. Further, the medical network manager 100 may delegate the authority assigned to the medical network endpoint 200 to another medical network endpoint 200.

Further, the medical network manager 100 may receive a request for any of various types of processes and perform searching, matching, or the like for a subject to whom the authority to execute the process is granted (for example, a doctor or a nurse associated with the medical network endpoint 200). As a specific example, the medical network manager 100 may receive a request related to execution of the process related to the medical practice and specify the medical network endpoint 200 to which the authority to execute the process related to the medical practice is granted (or the doctor or the nurse associated with the medical network endpoint 200) on the basis of a license required for implementing the medical practice or information of a field or the like of the medical practice or the like. Further, examples of the field of the medical practice include the field of medical treatment, specialty fields, and the like.

Further, the medical network manager 100 may be configured to be able to cooperate with other medical network managers 100 via a network. For example, in the example illustrated in FIG. 1, a medical network manager 100a is configured to be able to cooperate with another medical network manager 100b (for example, the medical network manager 100 of another medical institution) via a network.

The medical network information search database 400 is an entity for searching for information necessary for decision making of the medical network manager 100 and notifying the medical network manager 100 of a search result. The medical network information search database 400 may maintain a storage region (such as a repository or the like) for storing information serving as a search target. Further, the medical network information search database 400 may be configured as an information processing device such as a server and connected to the medical network manager 100 via a network. Further, as another example, the medical network information search database 400 may be installed in the medical network manager 100. In this case, a function corresponding to the medical network information search database 400 may be installed in the medical network manager 100 as software.

The medical network endpoint 200 is an entity which is responsible for an endpoint of the medical service provided via the network (hereinafter also referred to as a “medical network service”). As a specific example, the medical network endpoint 200 may be configured as a terminal device held by a patient. In this case, for example, the patient can use a remote diagnosis service via the medical network endpoint 200.

Further, as another example, the medical network endpoint 200 may be configured as a handset device operated by a doctor. In this case, the doctor can conduct a medical examination, diagnosis, or the like for a patient at a remote site via the medical network endpoint 200.

Further, as another example, a medical device installed in a medical institution such as a hospital may be configured as the medical network endpoint 200. Accordingly, for example, the doctor can access the medical device configured as the medical network endpoint 200 through another medical network endpoint 200 (for example, a terminal device) from a remote site via a network and operate the medical device. With such a configuration, for example, it is also possible to perform so-called remote surgery.

Further, one of the medical network endpoints 200 may operate as a master, and another medical network endpoint 200 may be connected to the medical network endpoint 200 as a slave. In this case, the medical network endpoint 200 operating as the master may manage an operation of the other medical network endpoint 200 connected to it as the slave.

The medical network enabler 300 is an entity which is responsible for a window of communication between the medical network endpoint 200 and the medical network manager 100. Specifically, the medical network enabler 300 performs processes such as translation, decryption, conversion, and the like in a format in which communication content of both the medical network endpoint 200 and the medical network manager 100 can be recognized. Further, the medical network enabler 300 may give a notification of content of a decision made by the medical network manager 100 to the user (for example, the doctor, the nurse, the patient, or the like) via a predetermined output unit (for example, a display of the medical network endpoint 200). Further, the medical network enabler 300 may have a function of causing the medical network endpoint 200 to be operated on the basis of the data notified of from the medical network manager 100.

In other words, it is desirable that the medical network enabler 300 be configured to be able to convert communication content between the medical network endpoint 200 and the medical network manager 100 into information having a predetermined format without impairing the meaning of the communication content. Further, the medical network enabler 300 may be configured as software to be installed in the medical network endpoint 200 provided as a physical device.

The medical network supervision database 500 is an entity which is installed for the purpose of monitoring whether or not an operation is performed in compliance with laws and ordinances is performed in a predetermined unit such as a country, a region, a medical group, or the like. The medical network supervision database 500 undertakes granting of authority to operate the medical network service, registration and management of information related to various types of network entities, authentication of various types of network entities, and the like.

The example of the architecture of the information processing system according to the present embodiment has been described above with reference to FIG. 1. Further, a more detailed process of various types of network entities illustrated in FIG. 1 will be separately described later together with an example assuming a specific application scene.

### 3. CONFIGURATION EXAMPLES

Next, as an example of a configuration of each network entity in the information processing system 1 according to the present embodiment, particularly, examples of functional configurations of the medical network manager 100 and the medical network endpoint 200 will be described.

### 3.1. Configuration Example of Medical Network Manager

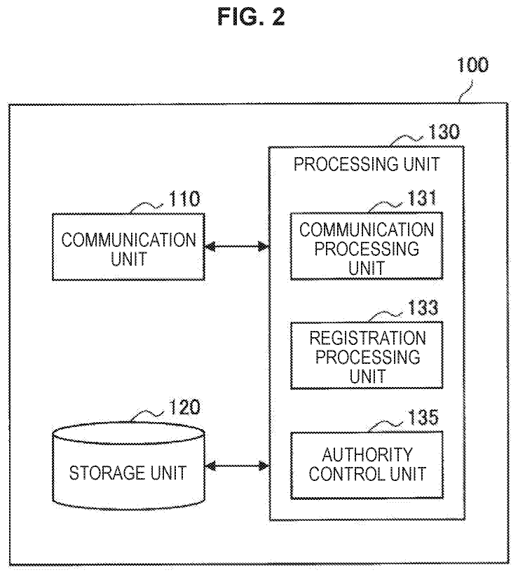

First, an example of a functional configuration of the medical network manager 100 according to the present embodiment will be described with reference to FIG. 2. FIG. 2 is a block diagram illustrating an example of a functional configuration of the medical network manager 100 according to the present embodiment.

As illustrated in FIG. 2, the medical network manager 100 includes a communication unit 110, a storage unit 120, and a processing unit 130.

The communication unit 110 is a component that enables each component of the medical network manager 100 to perform transmission and reception of information with another network entity (that is, another device, a service operating on a corresponding device, or the like) via a network. For example, the communication unit 110 transmits information to another network entity and receives information from the other network entity. The other network entity may include, for example, another medical network manager 100, the medical network endpoint 200, the medical network enabler 300, the medical network information search database 400, the medical network supervision database 500, and the like.

The storage unit 120 temporarily or permanently stores a program and various types of data for the operation of the medical network manager 100. Further, at least a part of the storage unit 120 may be configured as the medical network information search database 400.

The processing unit 130 provides various functions of the medical network manager 100. For example, the processing unit 130 includes a communication processing unit 131, a registration processing unit 133, and an authority control unit 135.

The communication processing unit 131 controls transmission and reception of information with another network entity via the network. For example, the communication processing unit 131 receives a request transmitted from another network entity, decrypts content of the request, and outputs a decryption result to the authority control unit 135 to be described later. Further, the communication processing unit 131 gives various types of notifications to another network entity. For example, the communication processing unit 131 may notify a network entity which is a transmission source of the request of a response to the request on the basis of various types of authentications results by the authority control unit 135 or a control result of a setting related to authority. Further, in the communication processing unit 131, a portion receiving the request corresponds to an example of a “receiving unit,” and a portion notifying of the response may correspond to an example of a “notifying unit.”

Further, the communication processing unit 131 may acquire information related to another network entity or information related to another target (for example, the user such as the doctor, the nurse, or the pharmacist) associated with the other network entity from the other network entity. In this case, the communication processing unit 131 may output the acquired information to the registration processing unit 133.

The registration processing unit 133 registers various types of information acquired from another network entity in the medical network information search database 400. Further, the registration processing unit 133 may also update the information registered in the medical network information search database 400 on the basis of various types of information acquired from another network entity.

The authority control unit 135 executes a process related to various types of authentications or control related to a setting of various types of authorities in accordance with content of the request from another network entity notified of from the communication processing unit 131. For example, the authority control unit 135 may perform authentication of whether or not execution of various types of processes by the network entity is permitted on the basis of attribute information directly or indirectly associated with another network entity in accordance with content of the notified request. Further, the information directly associated with the network entity includes, for example, attribute information related to the network entity itself. Further, the information indirectly associated with the network entity includes, for example, attribute information related to another object (for example, the user such as the doctor, the nurse, or the pharmacist) associated with the network entity. Further, the authority control unit 135 may correspond to an example of a “control unit” for controlling settings of various types of authorities and correspond to an example of an “authentication processing unit” for executing various types of authentication.

Further, as another example, the authority control unit 135 may control a setting of authority associated with execution of various types of processes for another network entity on the basis of the attribute information directly or indirectly associated with another network entity in accordance with the content of the notified request.

Further, the authority control unit 135 may acquire various types of information from a predetermined storage region (for example, the medical network supervision database 500) in accordance with the content of the notified request in order to execute the process related to various types of authentications or the control related to the settings of various types of authorities. For example, the authority control unit 135 may search for the medical network supervision database 500 on the basis of a condition indicated by the content of the notified request and extract information of the user (for example, the doctor, the nurse, the pharmacist, or the like) corresponding to the condition.

The example of the functional configuration of the medical network manager 100 according to the present embodiment has been described above with reference to FIG. 2. Further, the detailed content of the operation of the medical network manager 100 will be separately described later together with an example assuming a specific application scene.

### 3.2. Configuration Example of Medical Network Endpoint

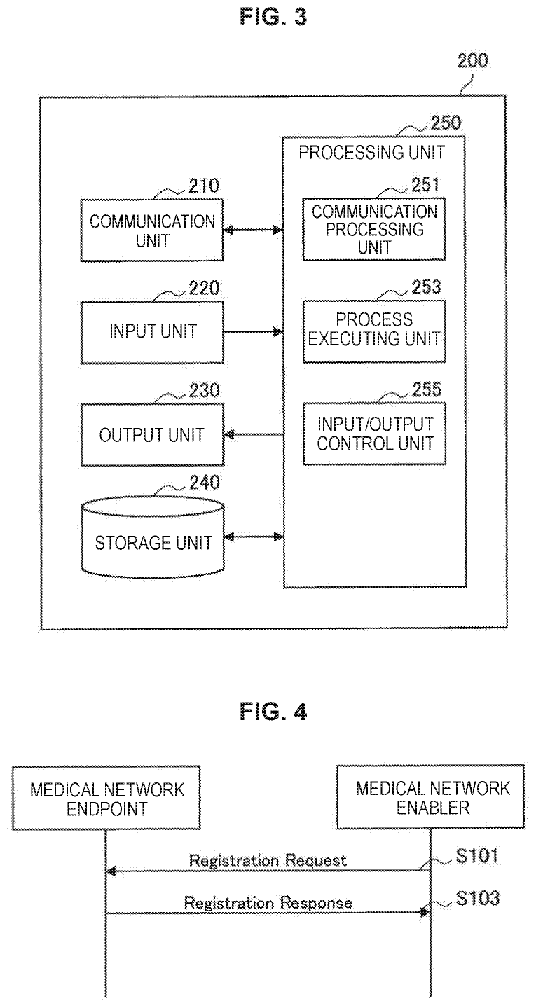

Next, an example of a functional configuration of the medical network endpoint 200 according to the present embodiment will be described with reference to FIG. 3. FIG. 3 is a block diagram illustrating an example of a functional configuration of the medical network endpoint 200 according to the present embodiment.

As illustrated in FIG. 3, the medical network endpoint 200 includes a communication unit 210, an input unit 220, an output unit 230, a storage unit 240, and a processing unit 250.

The communication unit 210 is a component that enables each component of the medical network endpoint 200 to perform transmission and reception of information with the medical network manager 100 via the network. For example, the communication unit 210 transmits information to the medical network manager 100 and receives information from the medical network manager 100.

The input unit 220 accepts an input from the user for the medical network endpoint 200 and outputs control information indicating the input contents to the processing unit 250. The input unit 220 may be constituted by an input device such as, for example, a mouse, a keyboard, a touch panel, a button, a switch, a lever, or a pedal.

The output unit 230 presents various types of information to the user. For example, the output unit 230 may be constituted by a display device such as a liquid crystal display or an electroluminescence (EL) display. In this case, the output unit 230 presents corresponding information to the user by displaying a screen on which information serving as a presentation target is presented. Further, the output unit 230 may be constituted by an acoustic device that outputs a sound such as a voice such as a speaker. In this case, the output unit 230 presents corresponding information to the user by outputting a voice or a sound corresponding to information to be presented. Further, the configuration of the output unit 230 is not limited to the above example as long as the information to be presented can be presented to the user in a predetermined form.

The storage unit 240 temporarily or permanently stores a program and various types of data for the operation of the medical network endpoint 200.

The processing unit 250 provides various functions of the medical network endpoint 200. For example, the processing unit 250 includes a communication processing unit 251, a process executing unit 253, and an input/output control unit 255. Further, the medical network enabler 300 associated with the medical network endpoint 200 may undertake at least some of the functions of the communication processing unit 251, the process executing unit 253, and the input/output control unit 255 which will be described below.

The communication processing unit 251 controls transmission and reception of information with the medical network manager 100 via the network. For example, the communication processing unit 131 transmits a request related to execution of various types of processes (for example, processes related to medical care) to the medical network manager 100. Further, the communication processing unit 251 may acquire various types of notifications from the medical network manager 100. For example, the communication processing unit 251 may acquire a response to the request from the medical network manager 100.

Further, the communication processing unit 251 may request the medical network manager 100 to register information of the medical network endpoint 200 or information related to another subject (for example, the user such as the doctor, the nurse, the pharmacist or the like) associated with the medical network endpoint 200. In this case, the communication processing unit 251 may read information to be registered (for example, the attribute information) from a predetermined storage region (for example, the storage unit 240). Further, as another example, the communication processing unit 251 may acquire it on the basis of a user input via the input unit 220. Further, in the communication processing unit 251, a portion for transmitting the request may correspond to an example of a “transmitting unit,” and a portion for acquiring the response may correspond to an example of an “acquiring unit.”

The process executing unit 253 is a component for executing various types of functions of the medical network endpoint 200. For example, the process executing unit 253 may execute a function (for example, an application) designated on the basis of the user input via the input unit 220 on the basis of various types of data stored in the storage unit 240. Further, the process executing unit 253 may execute a corresponding function on the basis of a notification from the medical network manager 100.

The input/output control unit 255 controls input of information via the input unit 220 and output of information via the output unit 230. For example, the input/output control unit 255 may present a UI for inputting information to the medical network endpoint 200 to the user via the output unit 230 and acquire an input result of the information to the UI from the input unit 220. Further, the input/output control unit 255 may present an execution result of various types of functions by the process executing unit 253 to the user via the output unit 230. Further, the input/output control unit 255 may also present output information based on a notification from the medical network manager 100 to the user via the output unit 230.

The example of the functional configuration of the medical network endpoint 200 according to the present embodiment has been described above with reference to FIG. 3.

### 4. TECHNICAL FEATURES

Next, technical features of the information processing system according to the present embodiment will be described.

First of all, in a case in which the provision of the medical services via the network is assumed as the premise, granting (delegation) of various types of authorities may occur. As a specific example, granting of an authority may occur under the following situation. Of course, the following examples are merely examples, and a situation in which granting of an authority may occur or a type of authority to be granted are not necessarily limited to examples illustrated below:
granting of an authentication or operation authority related to remote control of a medical device;
permission of a remote diagnosis action; and
an instruction related to a use of a medical examination device, presentation of a medical product, or the like from a doctor to a nurse in which emergency is assumed.

In the information processing system according to the present embodiment, a mechanism capable of realizing management of settings of various types of authorities (that is, granting of an authority, delegation of an authority, authentication, or the like) in a more suitable form in each of various situations in which grating or delegation of an authority occurs as described above is provided. Specifically, the information processing system according to the present embodiment realizes management of a settings of an authority according to various types of situations mainly on the basis of a “procedure related to registration of information” and a “procedure related to control of a setting of authority.” In this regard, technical features of the information processing system according to the present embodiment will be described below in the “procedure related to registration of information” and the “procedure related to control of a setting of authority.”

### 4.1. Procedure Related to Registration of Information

First, a process related to the procedure related to registration of information in the information processing system according to the present embodiment will be described. The procedure related to registration of information is a procedure in which, in the information processing system 1 according to the present embodiment, the network entity such as the medical network manager 100 collects, registers, and holds information serving as a material of decision making related to control of a setting of authority (for example, granting of an authority, delegation of an authority, authentication, or the like). The present procedure is performed between the network entities of the medical network endpoint 200, the medical network enabler 300, the medical network manager 100, and the medical network information search database 400. In this regard, an example of a flow of a series of processes will be described with reference to FIGS. 4 to 6 for each procedure related to registration of information performed between the network entities. FIGS. 4 to 6 are sequence diagrams illustrating an example of a flow of a series of processes for the procedure related to registration of information in the information processing system according to the present embodiment.

First, an example of a flow of a series of processes in the procedure between the medical network endpoint 200 and the medical network enabler 300 will be described with reference to FIG. 4.

The medical network enabler 300 requests the medical network endpoint 200 to present information to be registered by transmitting a request related to registration of information (S101). Upon receiving the request related to registration of information from the medical network enabler 300, the medical network endpoint 200 collects information to be registered and notifies the medical network enabler 300 of the collected information in association with a response to the request. Further, in the following description, the request is also referred to as an “information registration request (Registration Request),” and the response to the information registration request is also referred to as an “information registration response (Registration Response).”

Examples of the information to be registered include attribute information related to the medical network endpoint 200 itself and attribute information related to the user (for example, the doctor, the nurse, the pharmacist, patient, or the like) associated with the medical network endpoint 200. For example, Table 1 shown below indicates an example of information associated with the information registration response.

TABLE 1
Example of elements of registration response

| Parameter | Sub parameter | |
|---|---|---|
| name | name | Description |
| Endpoint | Serial number | Serial number assigned to an endpoint can be |
| specific | | included |
| information | Manufacturer | A manufacturer identifier of an endpoint can be |
| | identifier | included |
| | MAC address | MAC address information of an endpoint can |
| | | be included |
| Information | Country code | An identification code indicating a |
| related to laws | | country/region to which an endpoint belongs |
| and regulations | | can be included |
| | Authentication | Version information authenticated from a legal |
| | information | agency such as a hardware version, a firmware |
| | | version, or a software version can be included |
| User | User name | A name of an endpoint user can be included |
| information | User address | A contact address of an endpoint user can be |
| | | included |
| | User phone | A contact phone number of an endpoint user |
| | number | can be included |
| | User e-mail | A contact e-mail of an endpoint user can be |
| | address | included |
| | User category | An identifier indicating a type of an endpoint |
| | | user such as a doctor, a nurse, a pharmacist, or |
| | | a patient can be included |
| | User sex | A sex of an endpoint user can be included |
| | Insurance | A social security number or the like can be |
| | information | included |
| | (patient) | |
| | User language | Information related to a speaking language can |
| | information | be included |
| | Medical | Information related to an associated medical |
| | association | association can be included |
| | (doctor) | |
| | Associated | Information related to an associated medical |
| | medical | institution can be included |
| | institution | |
| License | Name | A name stated in a license can be included |
| information | Address | A registered address can be included |
| (doctor, nurse, | Number | A physician register number, a nurse license |
| pharmacist, | | number, or the like can be included |
| and the like) | Year of | A registration year can be included |
| | registration | |
| | Date of birth | A registered date of birth can be included |
| | Sex | A sex can be included |
| | Citizenship | Citizenship information can be included |
| | Medical | An identifier of whether or not a medical |
| | certificate | certificate related to his/her disability or the |
| | submission | like is submitted at time of license application |
| | identifier at time | |
| | of license | |
| | application | |
| Specialty | Field of medical | Information related to a field of medical |
| information | treatment | treatment classification can be included |
| (doctor) | classification | |
| | Field of specialty | Information related to a field of specialty |
| | classification | classification can be included |
| Position | GNSS | Information acquired by GNSS such as a |
| information | information | latitude, a longitude, and an altitude can be |
| | (mobile | included |
| | endpoint) | |
| | Installation | Information indicating a position of an |
| | position | endpoint or the like can be included |
| | information | |
| | (fixed type | |
| | endpoint) | |

Further, the information shown in Table 1 is merely an example, and the information to be registered is not necessarily limited to the example shown in Table 1. Further, instead of requiring all pieces of the information shown in Table 1, only some pieces of the information may be set as the registration target.

Further, as long as the medical network endpoint 200 can collect various types of information shown in Table 1, a method thereof is not particularly limited. For example, the medical network endpoint 200 may acquire the information to be registered with reference to data held in a predetermined storage region. Further, as another example, the medical network endpoint 200 may acquire the information to be registered on the basis of the user input via a predetermined input unit. Further, as another example, the medical network endpoint 200 may acquire the information to be registered from a device such as a predetermined server by accessing the device via the network.

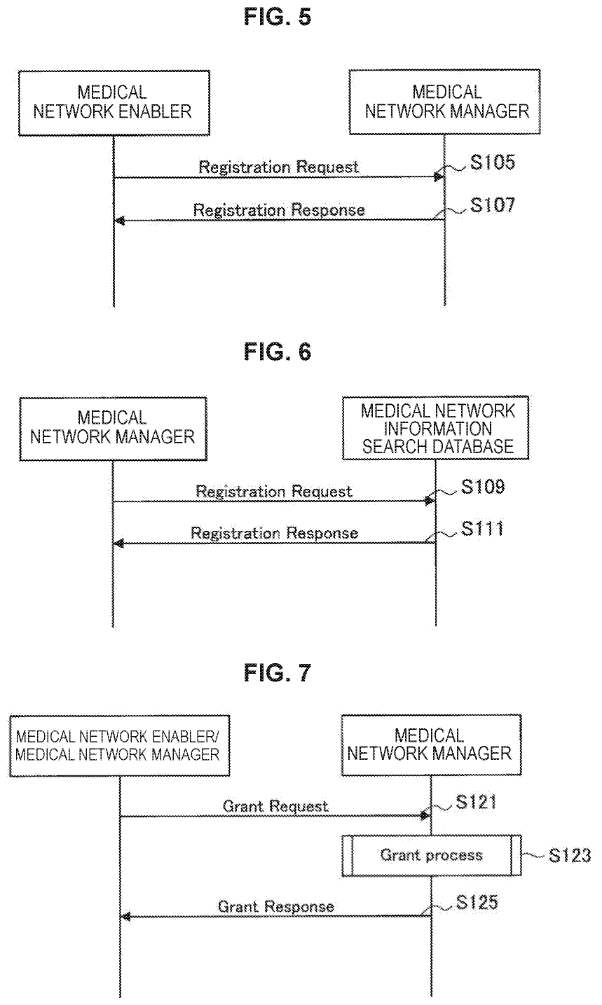

Next, an example of a flow of a series of processes in the procedure between the medical network enabler 300 and the medical network manager 100 will be described with reference to FIG. 5.

The medical network enabler 300 transmits a medical registration request to the medical network manager 100 after receiving a notification of the information registration response from the medical network endpoint 200 (S105). At this time, the medical network enabler 300 associates various types of information (for example, the information shown in Table 1) associated with the information registration response notified of from the medical network endpoint 200 with the information registration request transmitted to the medical network manager 100. Further, the medical network enabler 300 may additionally associate the information shown in the following Table 2 with the information registration request transmitted to the medical network manager 100.

TABLE 2
Example of elements of registration request (from enabler to manager)

| Parameter | Sub parameter | |
|---|---|---|
| Name | Name | Description |
| Enabler | Serial number | A serial number assigned to an enabler can be |
| specific | | included |
| information | Manufacturer | A manufacturer identifier of an enabler can be |
| | identifier | included |
| | MAC address | MAC address information of an enabler can be |
| | | included |
| Information | Country code | An identification code indicating a |
| related to laws | | country/region to which an enabler belongs can |
| and regulations | | be included |
| | Authentication | Version information authenticated from a legal |
| | information | agency such as a hardware version, a firmware |
| | | version, or a software version can be included |
| Information | Medical group | In a case in which an enabler is provided by a |
| related to a | identifier | medical group, information identifying the |
| medical group | | medical group can be included |

Upon receiving the information registration request from the medical network enabler 300, the medical network manager 100 notifies the medical network enabler 300 of the information registration response (S107).

Next, an example of a flow of a series of processes in the procedure between the medical network manager 100 and the medical network information search database 400 will be described with reference to FIG. 6.

Upon receiving the information registration request from the medical network enabler 300, the medical network manager 100 transmits the information registration request to the medical network information search database 400 (S109). At this time, the medical network manager 100 associates various types of information (for example, the information shown in Table 1 and Table 2) associated with the information registration response received from the medical network enabler 300 with the information registration request transmitted to the medical network information search database 400. Further, the medical network manager 100 may additionally associate information shown in the following Table 3 with the information registration request transmitted to the medical network information search database 400.

TABLE 3
Example of elements of registration request (from manager to database)

| Parameter | Sub parameter | |
|---|---|---|
| name | name | Description |
| Manager | Serial number | A serial number assigned to a manager can be |
| specific | | included |
| information | Manufacturer | A manufacturer identifier of a manager can be |
| | identifier | included |
| | MAC address | MAC address information of a manager can be |
| | | included |
| Information | Country code | An identification code indicating a |
| related to laws | | country/region to which a manager belongs can |
| and regulations | | be included |
| | Authentication | Version information authenticated from a legal |
| | information | agency such as a hardware version, a firmware |
| | | version, or a software version can be included |
| Information | Medical group | In a case in which a manager is provided by a |
| related to | identifier | medical group, information identifying the |
| medical group | | medical group can be included |

Upon receiving the information registration request from the medical network manager 100, the medical network information search database 400 causes various types of information associated with the information registration request to be stored in a predetermined storage region (for example, a repository). Then, the medical network information search database 400 notifies the medical network manager 100 of the information registration response (S111).

As described above, the example of the flow of a series of processing has been described with reference to FIGS. 4 to 6 for each procedure related to registration of information performed between the network entities. With the procedure described above, the network entity such as the medical network manager 100 can cause the information serving as a material for decision making related to control of a setting of authority (for example, granting or delegation of authority) to be held in the medical network information search database 400.

Further, the above description has proceed focusing on the procedures related to registration of information, but even in a case in which the information registered is updated with a change in various types of information, it is sufficient that updated information is transmitted to the medical network information search database 400 in accordance with a similar procedure. Further, in the above description, the procedures between the network entities has been individually described, but each procedure may be performed individually, or a plurality of procedures among at least some network entities may be sequentially performed as a series of procedures.

### 4.2. Procedure Related to Control of Setting of Authority

Next, a processing related to the procedure related to control of a setting of authority or the procedure related to various types of authentications in the information processing system according to the present embodiment will be described. In the information processing system 1 according to the present embodiment, control of a setting of authority such as granting or delegation of various types of authorities or various types of authentications are performed by the medical network manager 100 on the basis of various types of information registered in the medical network information search database 400. Further, in this description, the process of the information processing system according to the present embodiment will be described focusing on the process related to the control of the setting of the authority, and a basic concept (for example, a flow of a procedure between the network entities) of the process related to the authentication is similar, and thus detailed description thereof will be omitted.

### (1) First Case

For example, FIG. 7 is a sequence diagram illustrating a flow of a series of processes for an example of the procedure related to the control of the setting of the authority in the information processing system 1 according to the present embodiment. An example illustrated in FIG. 7 is an example in which the medical network manager 100 controls a setting of authority for the transmission source of the request on the basis of the request from the medical network enabler 300 or another medical network manager 100.

Specifically, as illustrated in FIG. 7, the medical network enabler 300 or another medical network manager 100 transmits a request related to granting or delegation of an authority to the medical network manager 100 (S121). Further, in the following description, the request is also referred to as an “authority setting request (Grant Request).”

Further, the medical network enabler 300 or another medical network manager 100 may associate information used for decision making by the medical network manager 100 with the authority setting request. For example, the following Table 4 shows an example of information associated with the authority setting request.

TABLE 4
Example of information which can be included in grant request

| Parameter | Sub parameter | |
|---|---|---|
| name | name | Description |
| Endpoint | Serial number | Serial number assigned to an endpoint |
| specific | | can be included |
| information | Manufacturer | A manufacturer identifier of an endpoint |
| | identifier | can be included |
| | MAC address | MAC address information of an endpoint |
| | | can be included |
| Enabler | Serial number | A serial number assigned to an enabler |
| specific | | can be included |
| information | Manufacturer | A manufacturer identifier of an enabler |
| | identifier | can be included |
| | MAC address | enabler MAC address information can be |
| | | included |
| Manager | Serial number | A serial number assigned to a manager |
| specific | | can be included |
| information | Manufacturer | A manufacturer identifier of a manager |
| | identifier | can be included |
| | MAC address | MAC address information of a manager |
| | | can be included |
| Position | GNSS | Information acquired by GNSS such as a |
| information | information | latitude, a longitude, and an altitude |
| | (mobile | can be included |
| | endpoint) | |
| | Installation | Information indicating a position at which |
| | position | an endpoint or the like is installed can |
| | information | be included |
| | (fixed type | |
| | endpoint) | |
| Authority | Authority | Information related to an authority which |
| information | identifier | is desired to be acquired can be |
| | Other | included Necessary other information may |
| | information | be included in accordance with an |
| | | authority which is desired to be acquired. |

Upon receiving the authority setting request from the medical network enabler 300 or another medical network manager 100, the medical network manager 100 performs decision making related to the control of the settings of the authority requested by the authority setting request. As a specific example, the medical network manager 100 causes the medical network information search database 400 to search for information related to a control target of a setting of authority on the basis of the information associated with the authority setting request and acquires the information. For example, the medical network manager 100 may acquire information related to the user corresponding to the medical network enabler 300 (for example, the user information, the license information, specialty information, or the like). Then, the medical network manager 100 performs decision making related to granting of an authority to the transmission source of the authority setting request on the basis of the acquired information, and performs the control of the setting of the authority for the transmission source of the authority setting request on the basis of the result (for example, granting of an authority) (S123).

Then, the medical network manager 100 notifies the transmission source of the authority setting request of the response to the authority setting request (S125). In the following description, the response is also referred to as an “authority setting response (Grant Response).”

Further, at this time, the medical network manager 100 may associate information related to the control result of the setting of the authority with the authority setting response. For example, Table 5 shown below shows an example of information associated with the authority setting response.

TABLE 5
Example of information which can be included in Grant response

| Parameter | Sub parameter | |
|---|---|---|
| name | name | Description |
| Authority | Grant authority | A code indicating granted authority can be |
| information | type code | included |
| | Authority | Information related to a validity period of an |
| | validity period | authority can be included |
| | Authority | Information of a manager who performs |
| | granter | determination of authority granting can be |
| | information | included |
| | Authority | Information of a manager/enabler/endpoint/user |
| | grantee | to which authority is granted can be included |
| | information | |
| | Other | Different necessary information can be |
| | information | included in accordance with grant authority |
| Authority grant | Status code | Status code can be included |
| status | | |
| Error | Error message | An error message can be included when an |
| classification | | error occurs |
| | Error data | Data such as insufficient information is |
| | | included depending on an error when an error |
| | | occurs |

As described above, in response to the request from the medical network enabler 300 or another medical network manager 100, by the medical network manager 100 controls the setting of the authority for the transmission source of the request on the basis of the attribute information directly or indirectly associated with the transmission source.

### (2) Second Case

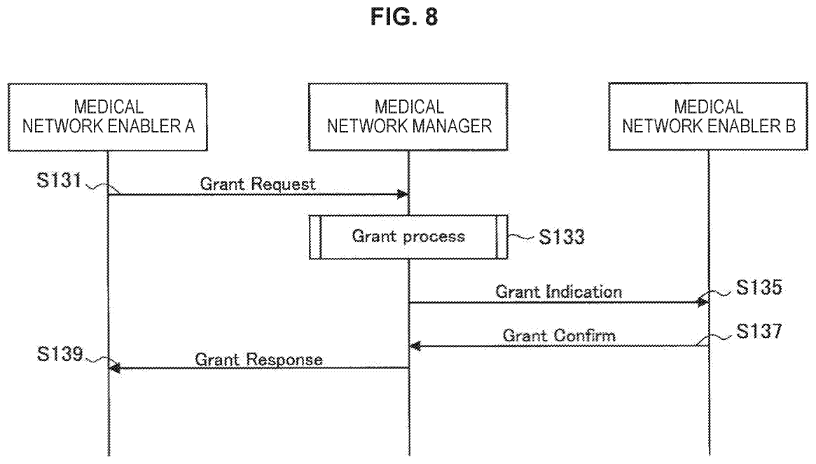

Next, another example of the procedure related to the control of the setting of the authority in the information processing system 1 according to the present embodiment will be described with reference to FIG. 8. FIG. 8 is a sequence diagram illustrating a flow of a series of processes for another example of the procedure related to the control of the setting of the authority in the information processing system 1 according to the present embodiment. An example illustrated in FIG. 8 is an example in which the medical network manager 100 controls the setting of the authority for another medical network enabler 300 on the basis of the request from the medical network enabler 300. Further, in this description, for the sake of convenience, the medical network enabler 300 which is the transmission source of the request is referred to as a “medical network enabler 300A,” and another medical network enabler 300 which is the control target of the setting of the authority is referred to as a “medical network enabler 300B.”

Specifically, as illustrated in FIG. 8, the medical network enabler 300A transmits the authority setting request (Grant Request) to the medical network manager 100 (S131). At this time, the medical network enabler 300A may associate the information used for decision making by the medical network manager 100 with the authority setting request. Further, the information associated with the authority setting request is information described above with reference to Table 1.

Upon receiving the authority setting request from the medical network enabler 300A, the medical network manager 100 performs decision making related to the control of the setting of the authority requested by the authority setting request. As a specific example, the medical network manager 100 causes the medical network information search database 400 to search for information related to the control target of the setting of the authority (that is, the medical network enabler 300B) on the basis of the information associated with the authority setting request and acquire the information. Further, for example, the medical network enabler 300B serving as the control target of the setting of the authority may be designated on the basis of the authority setting request by the medical network enabler 300A. Further, as another example, the medical network manager 100 may specify the medical network enabler 300B serving as the control target of the setting of the authority in accordance with the information associated with the authority setting request. Then, the medical network manager 100 performs decision making related to the control of the setting of the authority for the medical network enabler 300B on the basis of the acquired information, and performs the control of the setting of the authority for the medical network enabler 300B (for example, granting of the authority) (S133).

Then, the medical network manager 100 transmits a notification indicating that the control of the setting of the authority is performed to the medical network enabler 300B serving as the control target of the setting of the authority (S135). The medical network enabler 300B recognizes that the setting of the authority for itself is controlled on the basis of the notification from the medical network manager 100. Further, at this time, the medical network enabler 300B may cause the user to confirm whether or not the control result of the setting of the authority is accepted by presenting the control result of the setting of the authority to the user via the medical network endpoint 200 associated with itself. Then, the medical network enabler 300B transmits a confirmation result to the notification from the medical network manager 100 to the medical network manager 100 (S137). Further, in the following description, the notification is also referred to as an “authority setting notification (Grant Indication),” and the confirmation result transmitted as the response to the authority setting notification is also referred to as an “authority setting confirmation (Grant Confirm).”

Upon receiving the authority setting confirmation from the medical network enabler 300B as the response to the authority setting notification, the medical network manager 100 transmits the authority setting response to the medical network enabler 300A which is the transmission source of the authority setting request (S139),

As described above, in response to the request from the medical network enabler 300A, the medical network manager 100 controls the setting of the authority for the medical network enabler 300B on the basis of the attribute information directly or indirectly associated with the medical network enabler 300B. Further, in this case, the medical network manager 100 can also specify the medical network enabler 300B serving as the control target of the setting of the authority on the basis of the request from the medical network enabler 300A.

### (3) Third Case

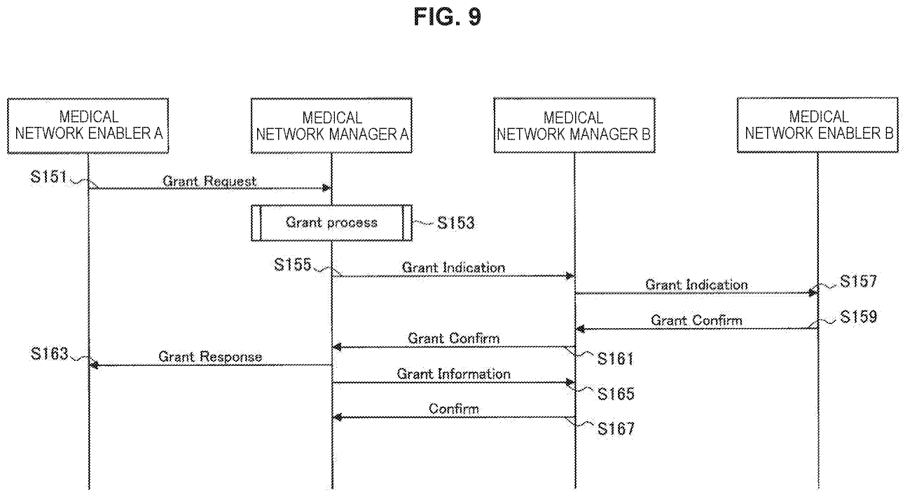

Next, another example of the procedure related to the control of the setting of the authority in the information processing system 1 according to the present embodiment will be described with reference to FIG. 9. FIG. 9 is a sequence diagram illustrating a flow of a series of processes for another example of the procedure related to the control of the setting of the authority in the information processing system 1 according to the present embodiment. An example illustrated in FIG. 9 is an example in which a plurality of medical network managers 100 operates in cooperation. Further, for the sake of convenience, the medical network enabler 300 which is the transmission source of the request is referred to as a “medical network enabler 300A,” and another medical network enabler 300 which is the control target of the setting of the authority is referred to as a “medical network enabler 300B.” Further, the medical network manager 100 managing the medical network enabler 300A is referred to as a “medical network manager 100A,” and the medical network manager 100 managing the medical network enabler 300B is referred to as a “medical network manager 100B.”

Specifically, as illustrated in FIG. 8, the medical network enabler 300A transmits the authority setting request (Grant Request) to the medical network manager 100A (S151). At this time, the medical network enabler 300A may associate the information used for decision making by the medical network manager 100A with the authority setting request. Further, the information associated with the authority setting request is information described above with reference to Table 1.

Upon receiving the authority setting request from the medical network enabler 300A, the medical network manager 100A performs decision making related to the control of the setting of the authority requested by the authority setting request. As a specific example, the medical network manager 100A causes the medical network information search database 400 to search for information related to the medical network enabler 300B corresponding to a condition corresponding to the information on the basis of the information associated with the authority setting request and acquires the information. Further, the medical network manager 100A may cause the medical network information search database 400 to acquire information related to the medical network manager 100B managing the medical network enabler 300B and acquire the information. Then, the medical network manager 100 performs decision making related to the control of the setting of the authority for the medical network enabler 300B on the basis of the acquired information, and performs the control of the setting of the authority for the medical network enabler 300B (for example, granting of the authority) (S153).

Then, the medical network manager 100A transmits the authority setting notification (Grant Indication) to the medical network manager 100B managing the medical network enabler 300B which is the control target of the setting of the authority (S155). At this time, the medical network manager 100A may associate information specifying the medical network enabler 300B serving as the control target of the setting of the authority with the authority setting notification.

Upon receiving the authority setting notification from the medical network manager 100A, the medical network manager 100B specifies the medical network enabler 300B serving as the control target of the setting of the authority on the basis of the authority setting notification. Then, the medical network manager 100B transfers the authority setting notification received from the medical network manager 100A to the specified medical network enabler 300B (S157). Further, at this time, the medical network enabler 300B may cause the user to confirm whether or not the control result of the setting of the authority is accepted by presenting the control result of the setting of the authority to the user via the medical network endpoint 200 associated with itself. Then, the medical network enabler 300B transmits the authority setting confirmation (Grant Confirm) to the medical network manager 100B as the response to the authority setting notification transmitted from the medical network manager 100B (S159). Upon receiving the authority setting confirmation from the medical network manager 100B as the response to the authority setting notification, the medical network manager 100B transfers the authority setting confirmation to the medical network manager 100A which is the transmission source of the authority setting notification (S161).

Upon receiving the authority setting confirmation from the medical network manager 100B as the response to the authority setting notification, the medical network manager 100A transmits the authority setting response (Grant Response) to the medical network enabler 300A which is the transmission source of the authority setting request (S163).

Further, the medical network manager 100A transfers information (Grant Information) related to the control of the setting of the authority such as information associated with the authority setting request or information acquired at the time of the control of the setting of the authority to the medical network manager 100B (S165). Upon receiving the information transferred from the medical network manager 100A, the medical network manager 100B gives a notification (Confirm) indicating that reception and confirmation of information are completed to the medical network manager 100A (S167).

With the above-described control, in response to the request from the medical network enabler 300A, the setting of the authority for the medical network enabler 300B managed by another medical network manager 100B is controlled on the basis of the attribute information associated directly or indirectly with the medical network enabler 300B. Accordingly, even under circumstances in which a plurality of medical institutions cooperates, it is possible to realize the management of settings of various types of authorities (that is, granting of an authority, delegation of an authority, authentication, or the like) in a more suitable form.

The process related to the procedure related to the control of the setting of the authority in the information processing system according to the present embodiment has been described below with reference to FIGS. 7 to 9.

### 5. EXAMPLES

Next, an example of the information processing system 1 according to the present embodiment will be described focusing on a more specific application scene of the information processing system 1.

### 5.1. First Example: Doctor-Patient Matching Service in Remote Diagnosis

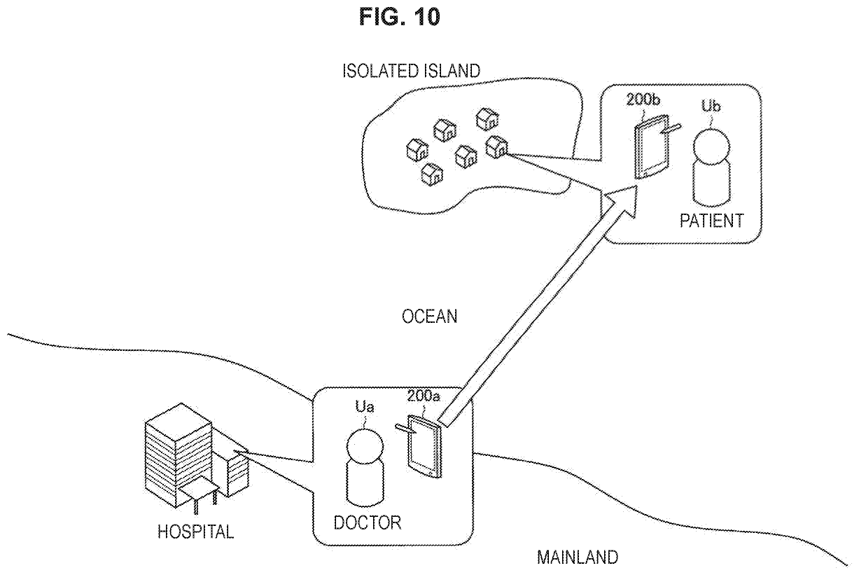

First, as a first example, an example of control for realizing a service of performing matching between a patient and a doctor in remote diagnosis in which the patient receives medical treatment from the doctor at a remote site via a network will be described. For example, FIG. 10 is an explanatory diagram for describing an overview of an information processing system according to the first example, and illustrates an example of a scene to which the above service is applied.

Specifically, as illustrated in FIG. 10, a doctor Ua in a hospital in a mainland provides a medical service (in particular, a medical examination service) via a network to a patient Ub living in an isolated island in which no doctor is resident at all times. Under such circumstances, if a feeling of a patient side is considered, there are cases in which it is desirable to cause the patient to have a contact point with a more excellent doctor who is as suitable for medical examination of a health state of the patient as possible. In light of this situation, the present example proposes an example of a mechanism for providing a matching service between a doctor and a patient using a network system.

First, as a premise, the doctor Ua registers information about him/herself in the medical network information search database 400 or a server accessible by the medical network information search database 400 via the medical network endpoint 200a held by him/herself. For example, Table 6 shown below shows an example of registration information related to a doctor.

TABLE 6
Example of registration information related to doctor

| Parameter | Sub parameter | |
|---|---|---|
| name | name | Value |
| Endpoint | Serial number | Abcdefg12345 |
| specific | Manufacturer | XXXX |
| information | identifier | |
| | MAC address | AA-BB-CC-DD-EE-FF-GG |
| Information | Country code | JP |
| related to laws | Authentication | Hardware ID: zzzzzzzz |
| and regulations | information | Software ID: xxxxxxxx |
| | | Firmware ID: yyyyyyyy |
| User | User name | Yamada Taro |
| information | User address | xx-yy-zz, Konan Minato-ku, |
| | | Tokyo |
| | User phone number | 03-47xx-89zz |
| | User e-mail address | Taro.Yamada@example.com |
| | User category | DR |
| | User sex | M |
| | User language | JP |
| | information | |
| | Medical association | SSS |
| | (doctor) | |
| | Associated medical | XYZ Hospital |
| | institution | |
| License | Name | Yamada Taro |
| information | Address | xx-yy-zz, Konan Minato-ku, |
| (doctor, nurse, | | Tokyo |
| pharmacist, | Number | 1532ss |
| and the like) | Year of registration | 2003 |
| | Date of birth | 197205xx |
| | Sex | M |
| | Citizenship | JP |
| | Medical certificate | TRUE |
| | submission identifier | |
| | at time of license | |
| | application | |
| Specialty | Field of medical | Internal medicine |
| information | treatment department | |
| (doctor) | classification | |
| | Field of specialty | Cardiology medicine |
| | classification | |
| | Installation position | xx-yy-zz, Konan Minato-ku, |
| | information (fixed | Tokyo |
| | type endpoint) | |

Similarly, the patient Ub registers information about him/herself in the medical network information search database 400 or a server accessible by the medical network information search database 400 via the medical network endpoint 200b held by him/herself. For example, Table 7 shown below shows an example of registration information related to a patient.

TABLE 7
Example of registration information related to patient

| Parameter | Sub parameter | |
|---|---|---|
| name | name | Description |
| Endpoint | Serial number | Efghi6789 |
| specific | Manufacturer | YYY |
| information | identifier | |
| | MAC address | HH-II-JJ-KK-LL-MM |
| Information | Country code | JP |
| related to laws | Authentication | Hardware ID: uuuuu |
| and regulations | information | Software ID: KKKKK |
| | | Firmware ID: GGGGG |
| User | User name | Hanako Yamada |
| information | User address | x-y-z, Osaki, Shinagawa-ku, Tokyo |
| | User phone | 080-59xy-85zz |
| | number | |
| | User e-mail | Hanako.Yamada@example.com |
| | address | |
| | User category | PA |
| | User sex | F |
| | Insurance | 63-13-xyz-A |
| | information | |
| | (patient) | |
| | User language | JP |
| | information | |
| Position | GNSS | 35.xxxx, 139.46ddd |
| information | information | |
| | (mobile | |
| | endpoint) | |

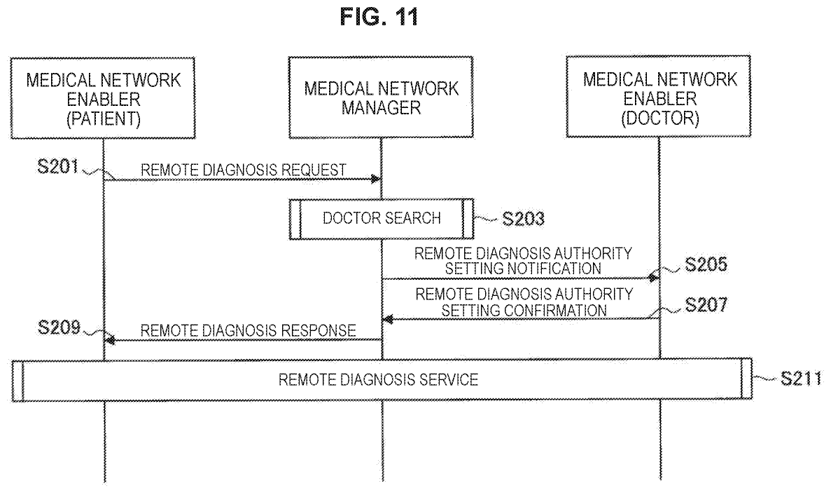

As described above, in the state in which the information of each of the doctor Ua and the patient Ub is registered, the patient Ub transmits a remote diagnosis request to the medical network manager 100 via the medical network endpoint 200b held by him/herself at a desired timing. For example, FIG. 11 is a sequence diagram illustrating an example of a flow of a series of processes of the information processing system 1 in accordance with the first example. Further, in this description, for the sake of convenience, the medical network enabler 300 associated with the medical network endpoint 200a held by the doctor Ua is also referred to as a “medical network enabler 300a.” Further, the medical network enabler 300 associated with the medical network endpoint 200b held by the patient Ub is also referred to as a “medical network enabler 300b.”

As illustrated in FIG. 11, if the patient Ub transmits the remote diagnosis request by operating the medical network endpoint 200b, the medical network enabler 300b transmits the remote diagnosis request to the medical network manager 100 (S201). At this time, the medical network enabler 300b may associate information used for decision making by the medical network manager 100 with the remote diagnosis request. Further, examples of the information associated with the remote diagnosis request include an “identifier indicating a remote diagnosis service” and “various types of information related to the remote diagnosis.” Further, examples of “various types of information related to the remote diagnosis” include a “type of a field of medical treatment,” a “desire for a sex of a doctor,” a “desired date of medical examination,” and “other comments.” Further, “other comments” indicate information which is arbitrarily input by the patient such as precautions.

Upon receiving the remote diagnosis request from the medical network enabler 300b, the medical network manager 100 causes the medical network information search database 400 to search for a doctor corresponding to a condition in accordance with the remote diagnosis request on the basis of the information associated with the remote diagnosis request. As a specific example, the medical network manager 100 may specify a doctor whose specialty information (that is, the field of medical treatment or the field of specialty) associated with each doctor as the attribute information satisfies a condition of the information associated with the remote diagnosis request. Further, as another example, the medical network manager 100 may specify a doctor (for example, a doctor located closer to the patient) who satisfies a condition of a distance between the doctor and the patient Ub on the basis of the position information associated with each doctor as the attribute information and the position information associated with the patient Ub as the attribute information. With such control, for example, the medical network manager 100 can acquire the information related to the doctor Ua satisfying a desire of the patient Ub or the medical network enabler 300a corresponding to the doctor Ua. Then, on the basis of the acquired information, the medical network manager 100 performs decision making related to granting of a remote diagnosis authority for the patient Ub to the medical network enabler 300a corresponding to the specified doctor Ua. Further, on the basis of the result of decision making, the medical network manager 100 performs control of the setting of the remote diagnosis authority for the patient Ub to the medical network enabler 300a (that is, granting of the remote diagnosis authority) (S203).

Next, the medical network manager 100 transmits the authority setting notification (remote diagnosis authority setting notification) indicating that granting of the setting of the remote diagnosis authority for the patient Ub has been performed to the medical network enabler 300a (S205). On the basis of the notification from the medical network manager 100, the medical network enabler 300a recognizes that the remote diagnosis authority for the patient Ub is granted to itself. Further, at this time, the medical network enabler 300a presents to the doctor Ua via the medical network endpoint 200a that the remote diagnosis authority is granted, and causes the doctor Ua to confirm whether or not the granting of the remote diagnosis authority for the patient Ub is accepted (that is, whether or not the remote diagnosis for the patient Ub is accepted). Then, If the acceptance of the granting of the remote diagnosis authority for the patient Ub by the doctor Ua is confirmed, the medical network enabler 300a transmits the authority setting confirmation (remote diagnosis authority setting confirmation) to the medical network manager 100 as the response to the remote diagnosis authority setting notification (S207).

Upon receiving the remote diagnosis authority setting confirmation from the medical network enabler 300a as the response to the remote diagnosis authority setting notification, the medical network manager 100 transmits the remote diagnosis response to the medical network enabler 300b (S209).

Accordingly, in response to the request for the remote diagnosis via the medical network endpoint 200b from the patient Ub, the doctor Ua is specified, an authority of the remote diagnosis for the patient Ub is granted to the medical network enabler 300a corresponding to the doctor Ua,

With such control, for example, the doctor Ua satisfying the condition (for example, a license, a field of medical practice, or the like) for performing the medical examination for the patient Ub is specified, and the authority to perform the remote diagnosis for the patient Ub is set to the doctor Ua in a more suitable form (for example, an authority is set within a necessary range). Accordingly, the doctor Ua can provide the remote diagnosis service to the patient Ub via the medical network endpoint 200a associated with the medical network enabler 300a (S211).

Further, although the present example has been described focusing on the example in which the remote diagnosis service is provided to the patient, a type of medical service is not particularly limited as long as the information processing system 1 can provide the medical service to the patient via the medical network endpoint 200. Further, the medical network manager 100 may control the medical service to be provided to the patient in accordance with the attribute information associated with the patient. As a specific example, the medical network manager 100 may switch a type of medical service to be provided to the patient, contents of the medical service selectively in accordance with the position information of each patient (that is, the position information of the medical network endpoint 200 associated with the patient).

As described above, an example of the control for realizing a service of performing matching between the patient and the doctor in the remote diagnosis in which the patient receives the medical examination from the doctor at a remote site via the network has been described with reference to FIGS. 10 and 11 as the first example.

### 5.2. Second Example: Remote Control of Medical Device

Next, an example in which, in a case in which a doctor performs remote control for a medical device via a network, control of a setting of authority related to the remote control for the medical device for the doctor is performed in response to a request from the doctor will be described as a second example.

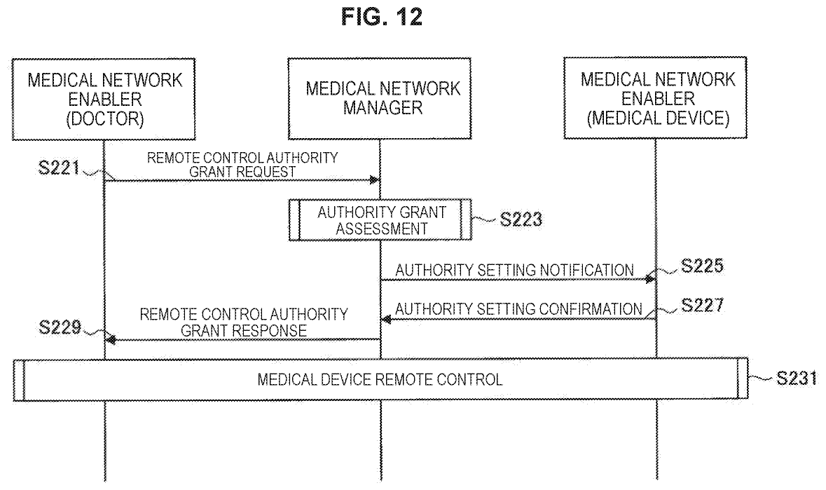

For example, FIG. 12 is a sequence diagram illustrating an example of a flow of a series of processes of an information processing system 1 according to the second example. Further, in this description, for the sake of convenience, the medical network endpoint 200 held by the doctor Ua is also referred to as a “medical network endpoint 200a.” Further, the medical network enabler 300 associated with the medical network endpoint 200a is also referred to as a “medical network enabler 300a.” Further, the medical network enabler 300 associated with the medical device is also referred to as a “medical network enabler 300c.”

As illustrated in FIG. 12, if the doctor Ua makes a request for granting an authority related to remote control for a medical device via the medical network endpoint 200a, the medical network enabler 300a transmits a remote control authority grant request to the medical network manager 100 (S221). At this time, the medical network enabler 300a may associate information used for decision making by the medical network manager 100 with the remote control authority grant request.

Upon receiving the remote control authority grant request from the medical network enabler 300a, the medical network manager 100 performs decision making related to granting of the authority of the remote control to the medical network enabler 300a. As a specific example, the medical network manager 100 causes the medical network information search database 400 to search for information related to the medical network enabler 300a or the doctor Ua associated with the medical network enabler 300a on the basis of the information associated with the remote control authority grant request and acquires the information. Further, the medical network manager 100 may cause the medical network information search database 400 to search for information related to a medical device to be subjected to the remote control or the medical network enabler 300c associated with the medical device and acquire the information. Further, the medical network manager 100 may specify the medical device to be subjected to the remote control or the medical network enabler 300c associated with the medical device on the basis of the information associated with the remote control authority grant request. Then, the medical network manager 100 performs decision making related to granting of an authority related to an operation of the medical device to the medical network enabler 300a on the basis of the acquired information. Further, on the basis of a result of decision making, the medical network manager 100 performs control of a setting of authority related to the remote control for the medical device to the medical network enabler 300a (for example, granting of an authority) (S223).

Then, the medical network manager 100 transmits an authority setting notification indicating that the authority related to the remote control for the medical device is granted to the medical network enabler 300a to the medical network enabler 300c associated with the medical device (S225). On the basis of the notification from the medical network manager 100, the medical network enabler 300c recognizes that the authority related to the remote control for the medical device associated with itself is granted to the medical network enabler 300a. Further, at this time, the medical network enabler 300c may perform access control on the medical device from the medical network enabler 300a. Then, the medical network enabler 300c transmits an authority setting confirmation to the authority setting notification from the medical network manager 100 to the medical network manager 100 (S227).

Upon receiving the authority setting confirmation as the response to the authority setting notification related to the remote control for the medical device from the medical network enabler 300c, the medical network manager 100 transmits a remote control authority grant response to the medical network enabler 300a (S229).

Accordingly, in response to the request from the doctor Ua, the authority related to the remote control for the medical device is granted to the medical network enabler 300a corresponding to the doctor Ua. With such control, for example, the authority related to the remote control for the medical device is set to the doctor Ua in a more suitable form (for example, an authority is set within a necessary range) in accordance with the attribute information (for example, the license, the field of medical practice, or the like) directly or indirectly associated with the doctor Ua. Accordingly, the doctor Ua can perform the remote control for the medical device via the medical network endpoint 200a associated with the medical network enabler 300a (S231).

With reference to FIG. 12, an example in which, in a case in which a doctor performs remote control for a medical device via a network, control of a setting of authority related to the remote control for the medical device for the doctor is performed in response to a request from the doctor has been described above as a second example.

### 5.3. Third Example: Handling Authority Authentication of Powerful Drugs

Next, an example in which unlocking of a door of a management storehouse strictly managed such as a powerful drug management storehouse in which powerful drugs and the like are managed or a money safe is managed via a network in accordance with attribute information associated with a user who performs unlocking (for example, the doctor, the nurse, the pharmacist, or the like) will be described as a third example. Further, in the third example, a UI is installed in the powerful drug management storehouse in which the powerful drugs and the like are managed, and the user such as the doctor, the nurse, or the pharmacist is assumed to perform various types of operations related to authentication (for example, input of information related to authentication) through the UI in a case in which unlocking of the powerful drug management storehouse is performed.

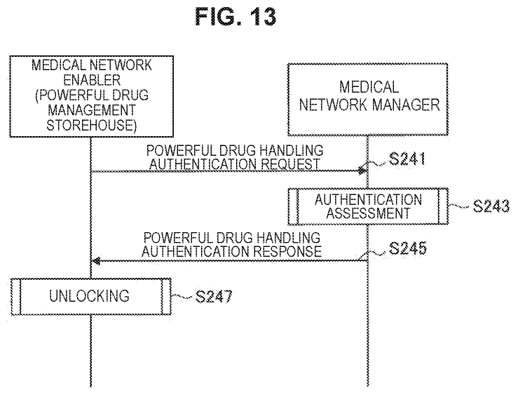

For example, FIG. 13 is a sequence diagram illustrating an example of a flow of a series of processes of an information processing system 1 according to the third example. Further, in this description, the medical network enabler 300 is associated with the powerful drug management storehouse, and for sake of convenience, the medical network enabler 300 is also referred to as a “medical network enabler 300d.”

For example, if the doctor inputs authentication information for unlocking the door of the powerful drug management storehouse to the UI of the powerful drug management storehouse, the medical network enabler 300d transmits a request for authentication related to handling of the powerful drug (powerful drug handling authentication request) to the medical network manager 100 (S241). At this time, the medical network enabler 300d associates the authentication information input via the UI with the powerful drug handling authentication request as information used for decision making by the medical network manager 100.

Upon receiving the powerful drug handling authentication request from medical network enabler 300d, the medical network manager 100 performs decision making related to unlocking of the door of the powerful drug management storehouse (that is, authentication assessment of the authority related to handling of the powerful drug) on the basis of the authentication information associated with the powerful drug handling authentication request. As a specific example, the medical network manager 100 causes the medical network information search database 400 to search for the information related to the user (for example, the doctor, the nurse, the pharmacist, or the like) indicated by the authentication information on the basis of the information associated with the remote control authority grant request and acquires the information. Next, the medical network manager 100 performs the authentication assessment of the authority related to the handling of the powerful drug for the user indicated by the authentication information on the basis of the acquired information (S243).

Then, the medical network manager 100 associates the information based on the result of the authentication assessment with a powerful drug handling authentication response, and transmits the powerful drug handling authentication response to the medical network enabler 300c (S245). At this time, in a case in which it is confirmed that the user indicated by the authentication information is not eligible for handling a desired powerful drug, the medical network manager 100 may decline unlocking of the door of the powerful drug management storehouse by the authentication response.

The medical network enabler 300c acquires the powerful drug handling authentication response from the medical network manager 100 as the response to the powerful drug handling authentication request, and recognizes the result of the authentication assessment on the basis of the acquired powerful drug handling authentication response. Then, it is sufficient if the medical network enabler 300c unlocks the door of the powerful drug management storehouse (that is, releases the lock) in accordance with the result of the authentication assessment (S247).

The example in which the unlocking of the door of the management storehouse strictly managed such as the powerful drug management storehouse or a money safe is managed via a network in accordance with the attribute information associated with the user who performs unlocking has been described above as the third example.

### 5.4. Fourth Example: Patient Watching and Handover by Nurse

Next, an example in which, in a situation in which the nurse watches over the patient, in a case in which the nurse hands the patient whom the nurse is responsible for over another nurse, control of settings of various types of authorities related to the patient (for example, granting or delegation of an authority or the like) is performed will be described with reference to FIG. 13 as a fourth example.

For example, a case in which there are also hospitalized patients which require more privacy protection than general hospitalized patients such as a VIP among hospitalized patients, and it is necessary to limit access to information to such hospitalized patients to some persons (for example, only nurses in charge) can be also assumed. In this regard, in the present example, an example of a mechanism for realizing management of hospitalized patients whom respective nurses are responsible for and handover between nurses associated with a change of a nurse in charge under the assumption of such circumstances will be described.

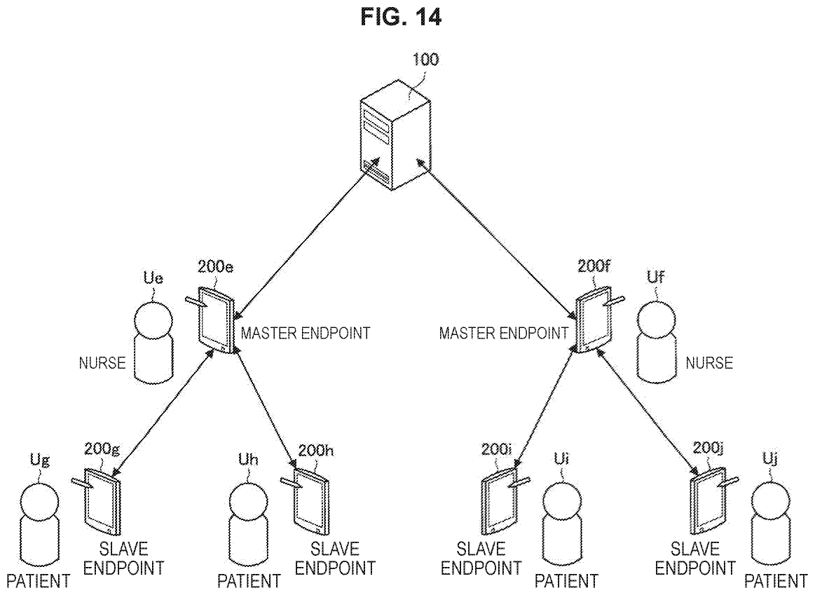

For example, FIG. 14 is an explanatory diagram for describing an example of a schematic configuration of an information processing system 1 according to the fourth example. As illustrated in FIG. 14, in the information processing system 1 according to the present example, the medical network endpoint 200 associated with each nurse operates as a master, and the medical network endpoint 200 associated with each patient operates as a slave. Further, the medical network endpoint 200 associated with a patient whom a certain nurse is responsible for watching over is connected to the medical network endpoint 200 associated with the nurse as the slave.

For example, in the example illustrated in FIG. 14, a nurse Ue is responsible for watching over patients Ug and Uh. Therefore, medical network endpoints 200g and 200h associated with the patients Ug and Uh are connected to a medical network endpoint 200e (master) associated with the nurse Ue as the slave. Similarly, a nurse Uf is responsible for watching over patients Ui and Uj. Therefore, medical network endpoints 200i and 200j associated with the patients Ui and Uj are connected to a medical network endpoint 200f (master) associated with the nurse Uf as the slave. With such a configuration, each nurse can manage information of the patient whom each nurse is responsible for via the medical network endpoint 200 (master) associated with the nurse. Further, it is also possible to restrict the nurse accessible to the information of the patient by limiting a connection relation between the medical network endpoint 200 associated with the nurse and the medical network endpoint 200 associated with the patient.

On the other hand, there are cases in which it is required to change a nurse in charge for a certain patient temporarily or permanently. As a specific example, in a case in which a certain nurse is absent due to off-duty or the like, a case in which watching over the patient whom the nurse is responsible for is handed over to another nurse can be assumed. In light of this situation, the information processing system 1 according to the present example provides a mechanism for realizing control of settings of various types of authorities related to a patient (for example, an authority to access information of a patient or the like) (for example, delegation of an authority) in a case in which a nurse responsible for a certain patient is changed.

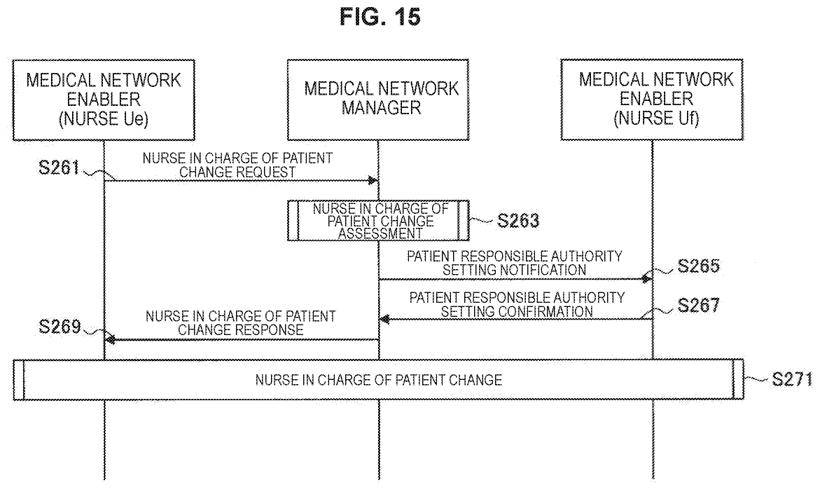

For example, FIG. 15 is a sequence diagram illustrating an example of a flow of a series of processes of the information processing system 1 according to the fourth example and illustrates an example in which settings of various types of authorities related to a patient are controlled with a change of a nurse responsible for a certain patient. Further, in this description, a case in which responsibility for the patient Ug is handed over from a nurse Ue to a nurse Uf will be described as an example. Further, in this description, for the sake of convenience, the medical network enabler 300 associated with the medical network endpoint 200e is also referred to as a “medical network enabler 300e.” Similarly, the medical network enabler 300 associated with the medical network endpoint 200f is also referred to as a “medical network enabler 300f.”

As illustrated in FIG. 15, if the nurse Ue makes a request related to a change of a nurse responsible for the patient Ug via the medical network endpoint 200e, the medical network enabler 300e transmits a nurse in charge of patient change request to the medical network manager 100 (S261). At this time, the medical network enabler 300e may associate the information related to the target patient Ug with the nurse in charge of patient change request. Further, the medical network enabler 300e may associate information related to nurse candidates after the nurse in charge is changed with the nurse in charge of patient change request. Further, the nurse candidates after the nurse in charge is changed may be designated by the nurse Ue via the medical network endpoint 200e or may be specified by the medical network enabler 300e on the basis of predetermined conditions.

Upon receiving the nurse in charge of patient change request from the medical network enabler 300e, the medical network manager 100 performs decision making (assessment) related to the change of the nurse in charge of the target patient Ug on the basis of the information associated with the nurse in charge of patient change request. As a specific example, the medical network manager 100 may cause the medical network information search database 400 to search for a nurse corresponding to a condition corresponding to the information on the basis of the information associated with the nurse in charge of patient change request. Further, as another example, in a case in which the information related to the nurse candidates after the nurse in charge is changed is associated with the nurse in charge of patient change request, the medical network manager 100 may cause the medical network information search database 400 to search for information related to each of the candidates (for example, attribute information of each candidate). Accordingly, the medical network manager 100 can specify the nurse after the nurse in charge is changed on the basis of the information related to each searched candidate. Then, the medical network manager 100 performs decision making related to granting of various types of authorities related to the patient Ug (for example, an authority to access information of the patient Ug) to the medical network enabler 300f corresponding to the specified nurse Uf after the nurse in charge is changed. Further, the medical network manager 100 performs control of settings of various types of authorities related to the patient Ug (for example, granting of an authority to access information of the patient Ug) to the medical network enabler 300f on the basis of a result of decision making (S263).

Then, the medical network manager 100 transmits an authority setting notification (patient responsible authority setting notification) indicating that various types of authorities related to the patient Ug are granted to the medical network enabler 300f corresponding to the nurse Uf after the nurse in charge is changed (S265). The medical network enabler 300f recognizes that various types of authorities related to the patient Ug are granted to itself on the basis of the patient responsible authority setting notification from the medical network manager 100. Further, at this time, the medical network enabler 300f may give a presentation indicating that various types of authorities related to the patient Ug are granted to the nurse Uf via the medical network endpoint 200f and cause the nurse Uf to confirm whether or not the granting of various types of authorities related to the patient Ug is accepted (that is, whether or not a role of the nurse in charge of the patient Ug is accepted). Then, if the acceptance of the granting of various types of authorities related to the patient Ug is confirmed by the nurse Uf, the medical network enabler 300f transmits an authority setting confirmation (patient responsible authority setting confirmation) to the medical network manager 100 as a response to the patient responsible authority setting notification (S267).

Upon receiving the patient responsible authority setting confirmation from the medical network enabler 300f as the response to the patient responsible authority setting notification, the medical network manager 100 transmits a nurse in charge of patient change response to the medical network enabler 300e (S269).

Accordingly, various types of authorities related to the patient Ug are granted to the nurse Uf, and thus the nurse in charge of the patient Ug can be changed from the nurse Ue to the nurse Uf (S271).

Further, in a case in which the change of the nurse in charge of the patient Ug is recognized, the information related to the patient Ug may be exchanged between the medical network enablers 300e and 300f. On the other hand, in a case in which the change of the nurse in charge of the patient Ug is not recognized due to a certain reason, exchange of the information related to the patient Ug between the medical network enablers 300e and 300f may be restricted. For example, such restriction may be performed by the medical network manager 100.

With the above control, it is possible to smoothly change various types of authorities related to the patient while securing security by restricting access to information with high confidentiality to a relevant person (for example, nurses before and after the nurse in charge is changed) even in a situation in which a nurse in charge of a certain patient is changed.

As described above, the example in which, in a situation in which the nurse watches over the patient, in a case in which the nurse hands the patient whom the nurse is responsible for over another nurse, control of settings of various types of authorities related to the patient is performed has been described as the fourth example.

### 5.5. Fifth Example: Transfer of Hospitalized Patient

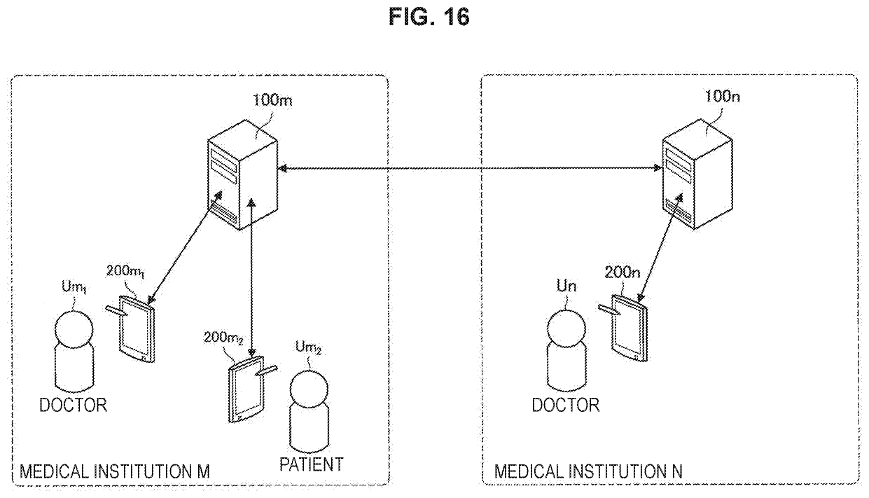

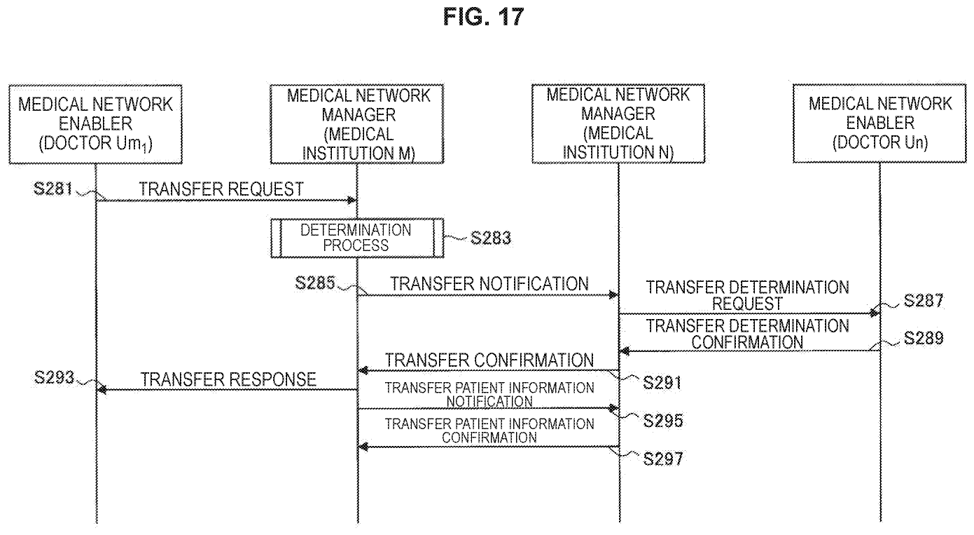

Next, an example of a mechanism for realizing a procedure related to transfer of a hospitalized patient between medical institutions via a network will be described with reference to FIGS. 16 and 17 as a fifth example. FIG. 16 is an explanatory diagram for describing an example of a schematic configuration of an information processing system 1 according to the fifth example. Further, FIG. 17 is a sequence diagram illustrating an example of a flow of a series of processes of the information processing system 1 according to the fifth example.

In the present example, an example of a configuration and a process of the information processing system 1 will be described focusing on a procedure between medical institution M and N using a case in which a patient Um₂ hospitalized to the medical institution M is caused to be transferred to the medical institution N as an example. Further, in this description, it is assumed that the medical network manager 100 is individually installed in each of the medical institution M and N. In this regard, for the sake of convenience, the medical network manager 100 corresponding to the medical institution M is also referred to as a “medical network manager 100m,” and the medical network manager 100 corresponding to the medical institution N is also referred to as a “medical network manager 100n.” Further, in this description, the medical network endpoint 200 associated with a doctor Um₁ belonging to the medical institution M is also referred to as a “medical network endpoint 200m₁.” Similarly, the medical network endpoint 200 associated with a doctor Un belonging to the medical institution N is also referred to as a “medical network endpoint 200n.” Further, the medical network enabler 300 associated with the medical network endpoint 200m₁ is also referred to as a “medical network enabler 300m₁.” Similarly, the medical network enabler 300 associated with the medical network endpoint 200n is also referred to as a “medical network enabler 300n.”

As illustrated in FIG. 17, the doctor Um₁ of the medical institution M is assumed to make a request related to the transfer of the patient Um₂ via the medical network endpoint 200m₁ associated with itself. Upon receiving this request, the medical network enabler 300m₁ transmits a transfer request to the medical network manager 100m (S281). At this time, the medical network enabler 300m₁ may associate various types of information such as information related to the patient Um₂ serving as a subject (for example, information of a field in which the patient Um₂ receive a medical examination or the like) with the transfer request.

Upon receiving the transfer request from the medical network enabler 300m₁, the medical network manager 100m performs decision making (determination) related to the transfer of the patient Um₂ serving as a subject on the basis of the information associated with the transfer request. As a specific example, the medical network manager 100m may cause the medical network information search database 400 to search for a medical institution corresponding to a condition corresponding to the information (that is, a medical institution serving as a transfer destination) on the basis of the information associated with the transfer request. As a more specific example, the medical network manager 100m may specify a medical institution which can handle the field in which the patient Um₂ receives the medical examination (for example, a medical institution which a doctor who can be responsible for the field belongs to). Further, the medical network manager 100m may cause the medical network information search database 400 to search for a medical institution serving as a transfer destination (or a candidate of the medical institution) on the basis of information related to the medical institution M associated with itself. As a specific example, the medical network manager 100m may specify a medical institution located near the medical institution M. Then, the medical network manager 100m performs decision making related to the transfer of the patient Um₂ to the specified medical institution N (for example, determination of whether or not it is possible to transfer) (S283).

Then, the medical network manager 100m makes requests to determine whether or not the patient Um₂ can be accepted by transmitting a transfer notification to the medical network manager 100n corresponding to the medical institution N in accordance with the result of decision making (S285). At this time, the medical network manager 100m may associate the information related to the patient Um₂ with the transfer notification.

Upon receiving the transfer notification from the medical network manager 100m, the medical network manager 100n determines whether or not the patient Um₂ serving as the subject can be accepted. At this time, the medical network manager 100n may determine whether or not the patient Um₂ can be accepted on the basis of the information associated with the transfer notification.

Further, the medical network manager 100n may transmit a determination request for the transfer of the patient Um₂ to the doctor Un belonging to the medical institution N. Specifically, the medical network manager 100n transmits a transfer determination request to the medical network enabler 300n corresponding to the doctor Un (S287). At this time, the medical network manager 100n may associate information related to the patient Um₂ with the transfer determination request. On the basis of the transfer determination request from the medical network manager 100n, the medical network enabler 300n causes the doctor Un to confirm whether or not the patient Um₂ can be accepted via the medical network endpoint 200n. Further, if the medical network enabler 300n acquires a determination result of the acceptance of the patient Um₂ by the doctor Un via the medical network endpoint 200n, the medical network enabler 300n transmits a transfer determination confirmation to the medical network manager 100 as a response to the transfer determination request (S289). In this case, the medical network manager 100n may recognize whether or not the patient Um₂ can be accepted on the basis of the transfer determination confirmation from the medical network endpoint 200n.

Then, the medical network manager 100n transmits a transfer confirmation based on a determination result of whether or not the patient Um₂ can be accepted to the medical network manager 100m as a response to the transfer notification (S291). Accordingly, the medical network manager 100m can recognize whether or not the medical institution N can accept the patient Um₂ on the basis of the transfer confirmation.

Upon receiving the transfer confirmation from the medical network manager 100n as the response to the transfer notification, the medical network manager 100m transmits a transfer response corresponding to the transfer confirmation to the medical network enabler 300m₂ (S293).

Further, in a case in which the medical institution N can accept the patient Um₂, the medical network manager 100m may notify the medical network manager 100n of information related to the patient Um₂ as transfer patient information (S295). Further, in a case in which the medical network manager 100n receives a notification of the information related to the patient Um₂ as the transfer patient information from the medical network manager 100m, the medical network manager 100n may notify the medical network manager 100m of a transfer patient information confirmation (S297).

As described above, the example of the mechanism for realizing the procedure related to the transfer of the hospitalized patient between the medical institutions has been described with reference to FIGS. 16 and 17 as the fifth example.

### 5.6. Sixth Example: Supervision and Business Permission for Medical Institution by Supervising Institution

Next, an example of a mechanism for realizing a procedure of supervision and business permission for a medical institution supervision by a supervising institution of a country or a region via a network will be described as a sixth example.

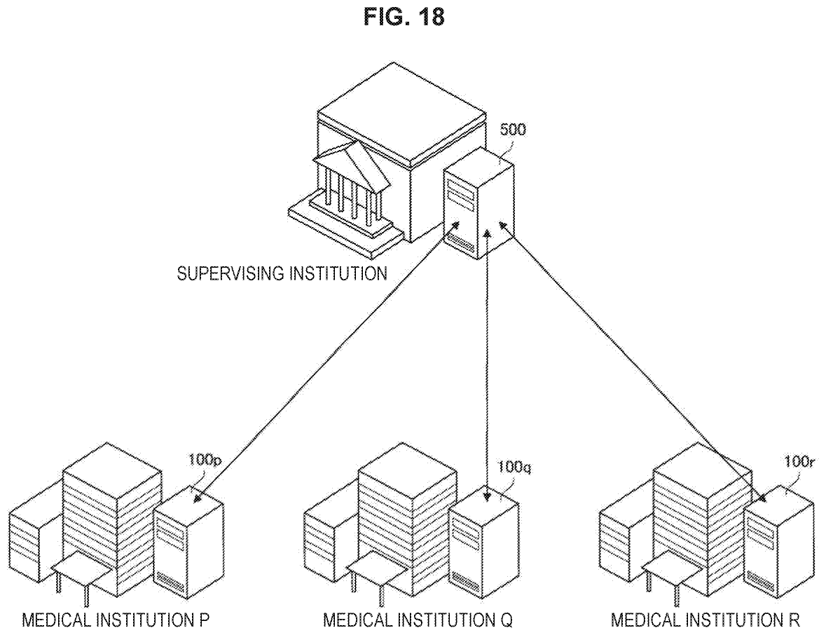

For example, FIG. 18 is an explanatory diagram for describing an example of a schematic configuration of an information processing system 1 according to the sixth example. As illustrated in FIG. 18, a medical network supervision database 500 is installed in the supervising institution. Further, the medical network supervision database 500 is configured to perform communication with medical network managers 100p to 100q respectively installed in medical institutions P to R via a network. Further, the medical network supervision database 500 has a function of managing various types of information and a function of making various types of decisions as in the medical network manager 100. On the basis of such a configuration, the medical network supervision database 500 performs approval determination related to registration of the medical institution, approval determination related to an audit after the registration, or the like on the basis of an application from the medical network manager 100 of each the medical institution. In this regard, in the following description, as an example of a process related to various types of procedures in the information processing system 1 according to the present example, “Approval procedure related to registration of medical institution” and “Procedure related to audit of medical institution” will be described.

### (1) Approval Procedure Related to Registration of Medical Institution

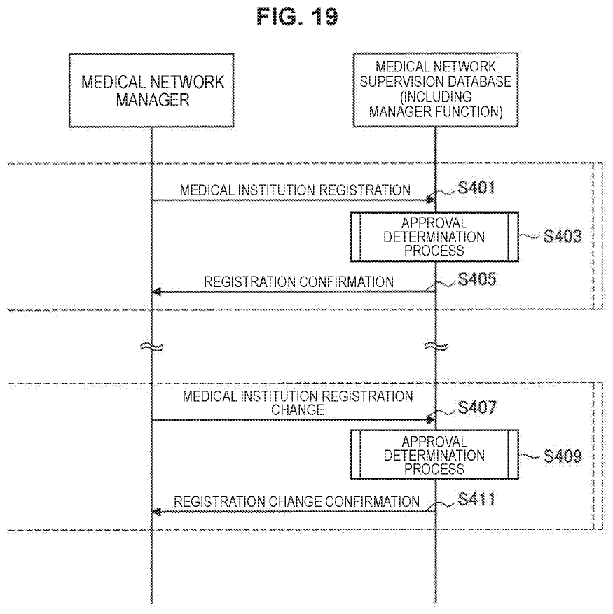

First, as an example of a flow of a series of processes of the information processing system 1 according to the sixth example, a process related to an approval procedure related to the registration of the medical institution will be described with reference to FIG. 19. FIG. 19 is a sequence diagram illustrating an example of a flow of a series of processes of the information processing system 1 according to the sixth example.

As illustrated in FIG. 19, the medical network manager 100 of each the medical institution applies for registration of a medical institution by transmitting various types of information related to the medical institution to the medical network supervision database 500 (S401). Upon receiving the application for the registration from the medical network manager 100, the medical network supervision database 500 performs approval determination for the medical institution on the basis of information related to the medical institution transmitted together (S403). Further, the medical network supervision database 500 may record the information related to the medical institution serving as a registration application target in a predetermined storage region (for example, a repository) in accordance with the result of the approval determination.

Then, the medical network supervision database 500 notifies the medical network manager 100 of information related to a result of the approval determination (registration confirmation) as a response to the application (S405). As described above, the approval determination of each the medical institution is performed by the medical network supervision database 500, and the information related to the medical institution is registered.

Further, the information of each medical institution registered by the medical network supervision database 500 may be changed on the basis of a predetermined procedure (hereinafter also referred to as a “registration change procedure”). Further, the registration change procedure is similar to the procedure related to the registration of the medical institution as illustrated in FIG. 19. In other words, the medical network manager 100 of each medical institution applies for a change of the information registered as the medical institution by transmitting various types of information (particularly, changed information) related to the medical institution to the medical network supervision database 500 (S407). Upon receiving the application for the change of the registration from the medical network manager 100, the medical network supervision database 500 performs approval determination for the medical institution again on the basis of the information related to the medical institution transmitted together (S409).

Further, the medical network supervision database 500 updates the registered information related to the corresponding medical institution in accordance with the result of the approval determination on the basis of the changed information transmitted together with the registration change application. Then, the medical network supervision database 500 notifies the medical network manager 100 of information related to a result of the approval determination (registration change confirmation) as a response to the application (S411). As described above, the approval determination for each medical institution is performed by the medical network supervision database 500, and the information related to the already registered medical institution is updated to the changed information.

### (2) Procedure Related to Audit of Medical Institution

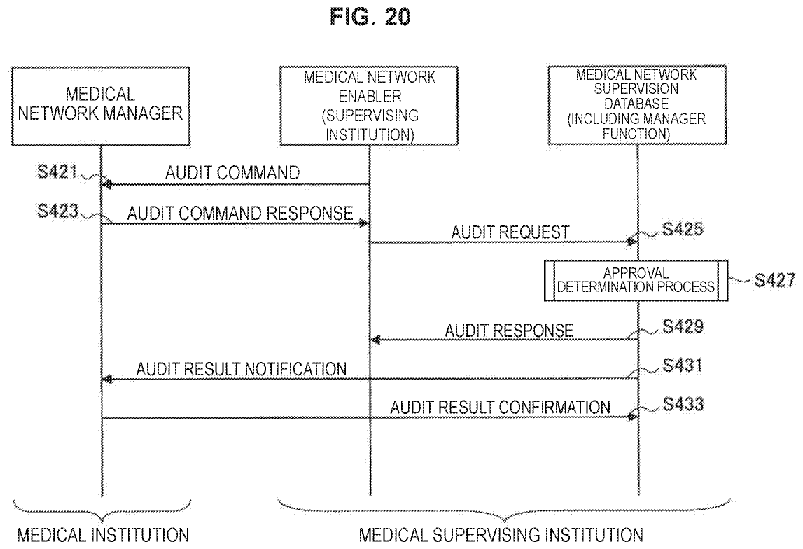

Next, as an example of a flow of a series of processes of the information processing system 1 according to the sixth example, a process related to the procedure related to the audit of the medical institution will be described with reference to FIG. 20. FIG. 20 is a sequence diagram illustrating an example of a flow of a series of processes of the information processing system 1 according to the sixth example.

Further, in this description, various types of command (for example, an audit command) are assumed to be given from a person in charge on the supervising institution side to a medical institution serving as an audit target. Specifically, the medical network enabler 300 is associated with the medical network endpoint 200 held by the person in charge on the supervising institution side. Then, as illustrated in FIG. 20, the medical network enabler 300 corresponding to the person in charge on the supervising institution side gives a notification of the audit command to the medical network manager 100 associated with the medical institution serving as the audit target (S421),

Upon receiving the notification of the audit command from the medical network enabler 300, the medical network manager 100 transmits information related to the medical institution associated with itself (particularly, information serving as an audit target) to the medical network enabler 300 as a response (audit command response) to the audit command (S423).

Upon receiving the information related to the medical institution serving as the target as the audit command response from the medical network manager 100, the medical network enabler 300 transmits an audit request to the medical network supervision database 500 on the basis of the received information (S425). At this time, the medical network enabler 300 associates the information related to the medical institution serving as the target with the audit request, and transmits the information to the medical network supervision database 500.

Upon receiving the audit request from the medical network enabler 300, the medical network supervision database 500 performs approval determination related to the audit of the medical institution on the basis of the information related to the medical institution associated with the audit request (S427). At this time, the medical network supervision database 500 may refer to the information related to the medical institution which is previously registered. Then, the medical network supervision database 500 notifies the medical network enabler 300 of an audit response as a response to the audit request (S429). Accordingly, the medical network enabler 300 can recognize that the audit request is accepted by the medical network supervision database 500.

Then, the medical network supervision database 500 gives a notification of information indicating the audit result to the medical network manager 100 associated with the medical institution serving as the audit target in accordance with the result of the approval determination (S431). Accordingly, the medical network manager 100 can recognize the result of the audit for the medical institution associated with itself. Further, the medical network manager 100 may give a notification of an audit result confirmation to the medical network supervision database 500 as a response to the notification of the audit result (S433).

As the sixth example, as a sixth example, the example of the mechanism for realizing the procedure of the supervision and the business permission for the medical institution by the supervising institution of the country or the region via the network has been described with reference to FIGS. 18 to 20, focusing on, particularly, “Approval procedure related to registration of medical institution” and “Procedure related to audit of medical institution.”

### 6. HARDWARE CONFIGURATION

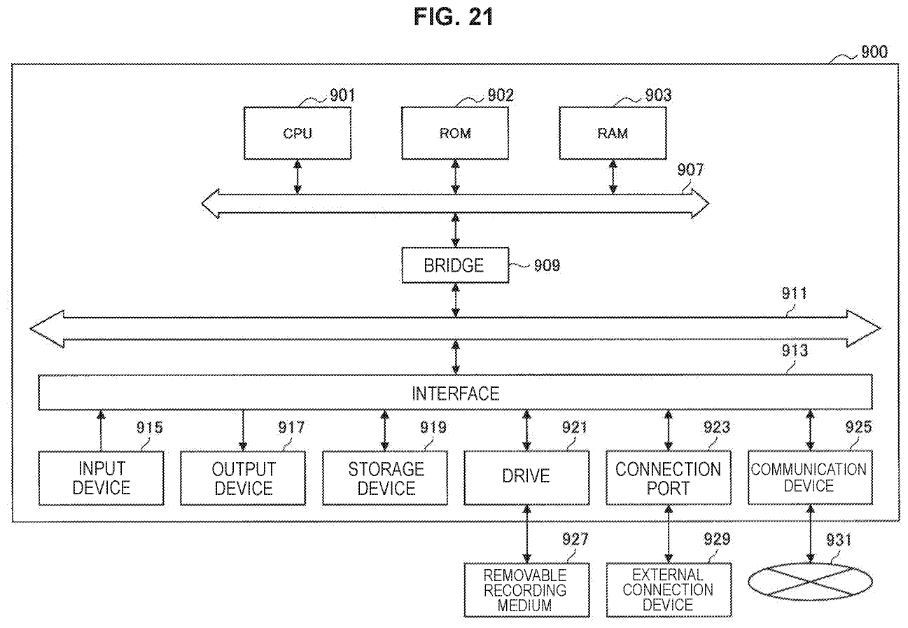

Next, a hardware configuration of an information processing device 900 such as the medical network manager 100 and the medical network endpoint 200 constituting the information processing system 1 according to an embodiment of the present disclosure will be described in detail with reference to FIG. 21. FIG. 21 is a functional block diagram illustrating an example of a hardware configuration of an information processing device constituting an information processing system according to an embodiment of the present disclosure.

The information processing device 900 included in the information processing system according to the present embodiment mainly includes a CPU 901, a ROM 903, and a RAM 905. Furthermore, the information processing device 900 also includes a host bus 907, a bridge 909, an external bus 911, an interface 913, an input device 915, an output device 917, a storage device 919, a drive 921, a connection port 923, and a communication device 925.

The CPU 901 serves as an arithmetic processing device and a control device, and controls the overall operation or a part of the operation of the information processing device 900 according to various programs recorded in the ROM 903, the RAM 905, the storage device 919, or a removable recording medium 927. The ROM 903 stores programs, operation parameters, and the like used by the CPU 901. The RAM 905 primarily stores programs that the CPU 901 uses and parameters and the like varying as appropriate during the execution of the programs. These are connected with each other via the host bus 907 including an internal bus such as a CPU bus or the like. Note that the processing unit 130 described above with reference to FIG. 2 and the processing unit 250 described above with reference to FIG. 3 can be realized by, for example, the CPU 901.

The host bus 907 is connected to the external bus 911 such as a PCI (Peripheral Component Interconnect/Interface) bus via the bridge 909. Additionally, the input device 915, the output device 917, the storage device 919, the drive 921, the connection port 923, and the communication device 925 are connected to the external bus 911 via the interface 913.

The input device 915 is an operation mechanism operated by a user, such as a mouse, a keyboard, a touch panel, buttons, a switch, a lever, or a pedal. Also, the input device 915 may be a remote control mechanism (a so-called remote control) using, for example, infrared light or other radio waves, or may be an external connection device 929 such as a mobile phone or a PDA conforming to the operation of the information processing device 900. Furthermore, the input device 915 generates an input signal based on, for example, information which is input by a user with the above operation mechanism, and includes an input control circuit for outputting the input signal to the CPU 901. The user of the information processing device 900 can input various data to the information processing device 900 and can instruct the information processing device 900 to perform processing by operating the input device 915. Note that the input unit 220 described above with reference to FIG. 3 can be realized by, for example, the input device 915.

The output device 917 includes a device capable of visually or audibly notifying acquired information to a user. Examples of such device include display devices such as a CRT display device, a liquid crystal display device, a plasma display device, an EL display device and lamps, audio output devices such as a speaker and a headphone, a printer, and the like. For example, the output device 917 outputs a result obtained by various processes performed by the information processing device 900. More specifically, the display device displays, in the form of texts or images, a result obtained by various processes performed by the information processing device 900. On the other hand, the audio output device converts an audio signal including reproduced audio data and sound data into an analog signal, and outputs the analog signal. Note that the output unit 230 described above with reference to FIG. 3 can be realized by, for example, the output device 917.

The storage device 919 is a device for storing data configured as an example of a storage unit of the information processing device 900. The storage device 919 is configured from, for example, a magnetic storage device such as a HDD (Hard Disk Drive), a semiconductor storage device, an optical storage device, or a magneto-optical storage device. This storage device 919 stores programs to be executed by the CPU 901, and various data. Note that the storage unit 120 described above with reference to FIG. 2 and the storage unit 240 described above with reference to FIG. 3 can be realized by, for example, the storage device 919.

The drive 921 is a reader/writer for recording medium, and is embedded in the information processing device 900 or attached externally thereto. The drive 921 reads information recorded in the attached removable recording medium 927 such as a magnetic disk, an optical disc, a magneto-optical disk, or a semiconductor memory, and outputs the read information to the RAM 905. Furthermore, the drive 921 can write record in the attached removable recording medium 927 such as a magnetic disk, an optical disc, a magneto-optical disk, or a semiconductor memory. The removable recording medium 927 is, for example, a DVD medium, an HD-DVD medium, or a Blu-ray (a registered trademark) medium. In addition, the removable recording medium 927 may be a CompactFlash (CF; a registered trademark), a flash memory, an SD memory card (Secure Digital Memory Card), or the like. Alternatively, the removable recording medium 927 may be, for example, an IC card (Integrated Circuit Card) equipped with a non-contact IC chip or an electronic appliance.

The connection port 923 is a port for allowing devices to directly connect to the information processing device 900. Examples of the connection port 923 include a USB (Universal Serial Bus) port, an IEEE1394 port, a SCSI (Small Computer System Interface) port, and the like. Other examples of the connection port 923 include an RS-232C port, an optical audio terminal, an HDMI (a registered trademark) (High-Definition Multimedia Interface) port, and the like. By the external connection device 929 connecting to this connection port 923, the information processing device 900 directly obtains various types of data from the external connection device 929 and provides various types of data to the external connection device 929.

The communication device 925 is a communication interface including, for example, a communication device for connecting to a communication network 931. The communication device 925 is, for example, a wired or wireless LAN (Local Area Network), Bluetooth (registered trademark), a communication card for WUSB (Wireless USB), or the like. Alternatively, the communication device 925 may be a router for optical communication, a router for ADSL (Asymmetric Digital Subscriber Line), a modem for various communications, or the like. This communication device 925 can transmit and receive signals and the like in accordance with a predetermined protocol such as TCP/IP on the Internet and with other communication devices, for example. The communication network 931 connected to the communication device 925 includes a network and the like, which is connected via wire or wirelessly, and may be, for example, the Internet, a home LAN, infrared communication, radio wave communication, satellite communication, or the like. Note that the communication unit 110 described with reference to FIG. 2 and the communication unit 210 described with reference to FIG. 3 can be realized by, for example, the storage device 919.

Heretofore, an example of the hardware configuration capable of realizing the functions of the information processing device 900 included in the information processing system according to the embodiment of the present disclosure has been shown. Each of the structural elements described above may be configured using a general-purpose material, or may be implemented by hardware dedicated to the function of each structural element. Accordingly, the hardware configuration to be used can be changed as appropriate according to the technical level at the time of carrying out the present embodiment. Note that, although not shown in FIG. 21, for example, it naturally includes various configurations corresponding to the information processing device 900 included in the information processing system according to the present embodiment.

Note that it is also possible to develop a computer program for realizing the respective functions of the information processing device 900 included in the information processing system according to the present embodiment as discussed above, and implement the computer program in a personal computer or the like. In addition, a computer-readable recording medium storing such a computer program may also be provided. The recording medium may be a magnetic disk, an optical disc, a magneto-optical disk, or flash memory, for example. Furthermore, the above computer program may also be delivered via a network, for example, without using a recording medium. In addition, the number of computers causing the computer program to be executed is not particularly limited. For example, the computer program may be executed in cooperation of a plurality of computers (for example, a plurality of servers or the like). Note that a single computer or a plurality of cooperating computers is also referred to as “computer system.”

### 7. CONCLUSION

As described above, in the information processing system 1 according to the present embodiment, the medical network manager 100 receives a request related to execution of a process associated with medical care from another device connected via a network (for example, the medical network endpoint 200 or another medical network manager 100). At this time, the medical network manager 100 may indirectly accept the request transmitted from a certain device (for example, the medical network endpoint 200) via another device (for example, another medical network manager 100). Then, the medical network manager 100 controls a setting of authority associated with execution of a process corresponding to the request for a device which is caused to execute the process or a user associate with the device (for example, a doctor, a nurse, a pharmacist, or the like) in accordance with attribute information directly or indirectly associated with the device. The attribute information includes, for example, attribute information related to a device which is caused to execute the process, attribute information related to the user associated with the device, and the like.

Further, in the information processing system 1 according to the present embodiment, the medical network manager 100 or the medical network supervision database 500 receives a request related to authentication of a process associated with medical care from another device connected via a network (for example, the medical network endpoint 200 or another medical network manager 100). At this time, the medical network manager 100 or the medical network supervision database 500 may receive the request transmitted from a certain device (for example, the medical network endpoint 200) indirectly via another device (for example, another medical network manager 100). Further, the medical network manager 100 or the medical network supervision database 500 executes the authentication corresponding to the authentication target in accordance with the attribute information directly or indirectly associated with the authentication target corresponding to the request.

With the above configuration, according to the information processing system according to the present embodiment, under circumstances in which various types of processes related to the medical care (for example, processes associated with the medical practice) are executed via a network, it is possible to realize management of a setting of authority to execute the process (for example, granting of an authority, delegation of an authority, authentication, or the like) in a more suitable form. Further, according to the information processing system according to the present embodiment, it is possible to restrict access to information with high confidentiality such as patient information, and thus it is possible to realize prompt transfer information between relevant persons while securing the confidentiality of information.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

An information processing device, including:

a receiving unit configured to receive a request related to execution of a process associated with medical care from a device directly or indirectly connected via a network; and

a control unit configured to control a setting of authority related to execution of the process for a device which is caused to execute the process corresponding to the request or a user associated with the device in accordance with attribute information directly or indirectly associated with the device.

(2)

The information processing device according to (1), in which the control unit controls a setting of the authority for a second device different from a first device which receives the request or a user associated with the second device in accordance with the attribute information associated directly or indirectly associated with the second device.

(3)

The information processing device according to (2), in which the control unit specifies the second device which is caused to execute the process corresponding to the request on the basis of information associated with the request and the attribute information directly or indirectly associated with each of the one or more second devices, and controls the setting of the authority for the specified second device or the user associated with the second device.

(4)

The information processing device according to (3), in which the attribute information includes position information of the second device or the user associated with the second device, and

the control unit specifies the second device which is caused to execute the process corresponding to the request on the basis of the position information corresponding to each of the one or more second devices.

(5)

The information processing device according to (3) or (4), in which the attribute information includes information related to medical practice as the information of the user associated with the second device, and

the control unit specifies the second device which is caused to execute the process corresponding to the request on the basis of the information associated with medical practice corresponding to the user associated with each of the one or more second devices.

(6)

The information processing device according to (5), in which the information associated with the medical practice includes information related to a license related to implementation of the medical practice of the user associated with the second device.

(7)

The information processing device according to (5) or (6), in which the information associated with the medical practice includes information related to at least one of a field of medical treatment or a field of specialty of the user associated with the second device.

(8)

The information processing device according to (1), in which the control unit controls a setting of the authority for a first device which receives the request or a user associated with the first device in accordance with the attribute information associated directly or indirectly with the first device.

(9)

The information processing device according to any one of (1) to (8), including: a notifying unit configured to give a notification indicating information related to a control result of the setting of the authority to the device which is caused to execute the process corresponding to the request.

(10)

The information processing device according to (9), in which the notifying unit gives a notification indicating information related to a control result of the setting of the authority to the device which is a transmission source of the request.

(11)

An information processing device, including:

a transmitting unit configured to transmit a request related to execution of a process associated with medical care to a device directly or indirectly connected via a network; and

an acquiring unit configured to acquire a control result of a setting of authority associated with execution of the process for the device or a user associated with the device, corresponding to attribute information directly or indirectly associated with the device which is caused to execute the process corresponding to the request after the request is transmitted.

(12)

An information processing device, including:

a receiving unit configured to receive a request related to authentication of a process associated with medical care from a device directly or indirectly connected via a network; and

an authentication processing unit configured to execute the authentication on an authentication target corresponding to the request in accordance with attribute information directly or indirectly associated with the authentication target.

(13)

The information processing device according to (12), including:

a notifying unit configured to give a notification indicating information related to a result of the authentication to the device which is a transmission source of the request.

(14)

An information processing device, including:

a transmitting unit configured to transmit a request related to authentication of a process associated with medical care to a device directly or indirectly connected via a network; and

an acquiring unit configured to acquire a result of the authentication on an authentication target corresponding to the request in accordance with attribute information directly or indirectly associated with the authentication target from the device after the request is transmitted.

(15)

An information processing method, including:

receiving, by a computer, a request related to execution of a process associated with medical care from a device directly or indirectly connected via a network; and

controlling, by the computer, a setting of authority related to execution of the process for a device which is caused to execute the process corresponding to the request or a user associated with the device in accordance with attribute information directly or indirectly associated with the device.

(16)

An information processing method, including:

transmitting, by a computer, a request related to execution of a process associated with medical care to a device directly or indirectly connected via a network; and

acquiring, by the computer, a control result of a setting of authority associated with execution of the process for the device or a user associated with the device, corresponding to attribute information directly or indirectly associated with the device which is caused to execute the process corresponding to the request from the device after the request is transmitted.

(17)

An information processing method, including:

receiving, by a computer, a request related to authentication of a process associated with medical care from a device directly or indirectly connected via a network; and

executing, by the computer, the authentication on an authentication target corresponding to the request in accordance with attribute information directly or indirectly associated with the authentication target.

(18)

An information processing method, including:

transmitting, by a computer, a request related to authentication of a process associated with medical care to a device directly or indirectly connected via a network; and

acquiring, by the computer, a result of the authentication on an authentication target corresponding to the request in accordance with attribute information directly or indirectly associated with the authentication target from the device after the request is transmitted.

(19)

A program causing a computer to execute:

receiving a request related to execution of a process associated with medical care from a device directly or indirectly connected via a network; and

controlling a setting of authority related to execution of the process for a device which is caused to execute the process corresponding to the request or a user associated with the device in accordance with attribute information directly or indirectly associated with the device.

(20)

A program causing a computer to execute:

transmitting a request related to execution of a process associated with medical care to a device directly or indirectly connected via a network; and

acquiring a control result of a setting of authority associated with execution of the process for the device or a user associated with the device, corresponding to attribute information directly or indirectly associated with the device which is caused to execute the process corresponding to the request from the device after the request is transmitted.

(21)

A program causing a computer to execute:

receiving a request related to authentication of a process associated with medical care from a device directly or indirectly connected via a network; and

executing the authentication on an authentication target corresponding to the request in accordance with attribute information directly or indirectly associated with the authentication target.

(22)

A program causing a computer to execute:

transmitting a request related to authentication of a process associated with medical care to a device directly or indirectly connected via a network; and

acquiring a result of the authentication on an authentication target corresponding to the request in accordance with attribute information directly or indirectly associated with the authentication target from the device after the request is transmitted.

### REFERENCE SIGNS LIST

1 information processing system
100 medical network manager
110 communication unit
120 storage unit
130 processing unit
131 communication processing unit
133 registration processing unit
135 authority control unit
200 medical network endpoint
210 communication unit
220 input unit
230 output unit
240 storage unit
250 processing unit
251 communication processing unit
253 process executing unit
255 input/output control unit
300 medical network enabler
400 medical network information search database
500 medical network supervision database

1. An information processing device, comprising:
a receiving unit configured to receive a request related to execution of a process associated with medical care from a device directly or indirectly connected via a network; and
a control unit configured to control a setting of authority related to execution of the process for a device which is caused to execute the process corresponding to the request or a user associated with the device in accordance with attribute information directly or indirectly associated with the device.

2. The information processing device according to claim 1, wherein the control unit controls a setting of the authority for a second device different from a first device which receives the request or a user associated with the second device in accordance with the attribute information associated directly or indirectly associated with the second device.

3. The information processing device according to claim 2, wherein the control unit specifies the second device which is caused to execute the process corresponding to the request on a basis of information associated with the request and the attribute information directly or indirectly associated with each of the one or more second devices, and controls the setting of the authority for the specified second device or the user associated with the second device.

4. The information processing device according to claim 3, wherein the attribute information includes position information of the second device or the user associated with the second device, and
the control unit specifies the second device which is caused to execute the process corresponding to the request on a basis of the position information corresponding to each of the one or more second devices.

5. The information processing device according to claim 3, wherein the attribute information includes information related to medical practice as the information of the user associated with the second device, and
the control unit specifies the second device which is caused to execute the process corresponding to the request on a basis of the information associated with medical practice corresponding to the user associated with each of the one or more second devices.

6. The information processing device according to claim 1, wherein the control unit controls a setting of the authority for a first device which receives the request or a user associated with the first device in accordance with the attribute information associated directly or indirectly with the first device.

7. The information processing device according to claim 1, comprising:
a notifying unit configured to give a notification indicating information related to a control result of the setting of the authority to the device which is caused to execute the process corresponding to the request.

8. The information processing device according to claim 7, wherein the notifying unit gives a notification indicating information related to a control result of the setting of the authority to the device which is a transmission source of the request.

9. An information processing device, comprising:
a transmitting unit configured to transmit a request related to execution of a process associated with medical care to a device directly or indirectly connected via a network; and
an acquiring unit configured to acquire a control result of a setting of authority associated with execution of the process for the device or a user associated with the device, corresponding to attribute information directly or indirectly associated with the device which is caused to execute the process corresponding to the request after the request is transmitted.

10. An information processing device, comprising:
a receiving unit configured to receive a request related to authentication of a process associated with medical care from a device directly or indirectly connected via a network; and
an authentication processing unit configured to execute the authentication on an authentication target corresponding to the request in accordance with attribute information directly or indirectly associated with the authentication target.

11. The information processing device according to claim 10, comprising:
a notifying unit configured to give a notification indicating information related to a result of the authentication to the device which is a transmission source of the request.

12. An information processing device, comprising:
a transmitting unit configured to transmit a request related to authentication of a process associated with medical care to a device directly or indirectly connected via a network; and
an acquiring unit configured to acquire a result of the authentication on an authentication target corresponding to the request in accordance with attribute information directly or indirectly associated with the authentication target from the device after the request is transmitted.

13. An information processing method, comprising:
receiving, by a computer, a request related to execution of a process associated with medical care from a device directly or indirectly connected via a network; and
controlling, by the computer, a setting of authority related to execution of the process for a device which is caused to execute the process corresponding to the request or a user associated with the device in accordance with attribute information directly or indirectly associated with the device.

14. An information processing method, comprising:
transmitting, by a computer, a request related to execution of a process associated with medical care to a device directly or indirectly connected via a network; and
acquiring, by the computer, a control result of a setting of authority associated with execution of the process for the device or a user associated with the device, corresponding to attribute information directly or indirectly associated with the device which is caused to execute the process corresponding to the request from the device after the request is transmitted.

15. An information processing method, comprising:
receiving, by a computer, a request related to authentication of a process associated with medical care from a device directly or indirectly connected via a network; and
executing, by the computer, the authentication on an authentication target corresponding to the request in accordance with attribute information directly or indirectly associated with the authentication target.

16. An information processing method, comprising:
transmitting, by a computer, a request related to authentication of a process associated with medical care to a device directly or indirectly connected via a network; and
acquiring, by the computer, a result of the authentication on an authentication target corresponding to the request in accordance with attribute information directly or indirectly associated with the authentication target from the device after the request is transmitted.

17. A program causing a computer to execute:
receiving a request related to execution of a process associated with medical care from a device directly or indirectly connected via a network; and
controlling a setting of authority related to execution of the process for a device which is caused to execute the process corresponding to the request or a user associated with the device in accordance with attribute information directly or indirectly associated with the device.

18. A program causing a computer to execute:
transmitting a request related to execution of a process associated with medical care to a device directly or indirectly connected via a network; and
acquiring a control result of a setting of authority associated with execution of the process for the device or a user associated with the device, corresponding to attribute information directly or indirectly associated with the device which is caused to execute the process corresponding to the request from the device after the request is transmitted.

19. A program causing a computer to execute:
receiving a request related to authentication of a process associated with medical care from a device directly or indirectly connected via a network; and
executing the authentication on an authentication target corresponding to the request in accordance with attribute information directly or indirectly associated with the authentication target.

20. A program causing a computer to execute:
transmitting a request related to authentication of a process associated with medical care to a device directly or indirectly connected via a network; and
acquiring a result of the authentication on an authentication target corresponding to the request in accordance with attribute information directly or indirectly associated with the authentication target from the device after the request is transmitted.
